(19) United States
(12) Patent Application Publication
(10) Pub. No.: US 2023/0141575 A1
(43) Pub. Date: May 11, 2023

(54) MULTIVALENT CHEMOKINE RECEPTOR BINDING COMPLEXES

(71) Applicant: CITY OF HOPE, Duarte, CA (US)

(72) Inventors: John C. WILLIAMS, Monrovia, CA (US); Miso PARK, Duarte, CA (US)

(21) Appl. No.: 17/906,467

(22) PCT Filed: Mar. 18, 2021
(86) PCT No.: PCT/US2021/023030

§ 371 Date: Sep. 15, 2022

Related U.S. Application Data
(60) Provisional application No. 62/991,388, filed on Mar. 18, 2020.

(51) Int. Cl. C07K 14/54; A61P 35/00

(52) U.S. Cl. CPC C07K 14/5443; A61P 35/00; C07K 2317/31; C07K 2317/55; C07K 2319/50; C07K 2319/30; A61K 38/00

(57) ABSTRACT

Provided herein are, inter alia, multi-specific ligand binding complexes capable of binding tumor-associated antigens and effector cell activating ligands. The complexes provided herein may include a first ligand binding domain (e.g., a Fab) capable of binding a tumor antigen. The complexes further include a second ligand binding domain (e.g., IL-15) non-covalently and/or covalently attached to a second ligand binding domain enhancer. The complexes provided herein are, inter alia, useful for the treatment of cancer.

### CROSS-REFERENCE TO RELATED APPLICATIONS

This application claims priority to U.S. Provisional Application No. 62/991,388, filed Mar. 18, 2020, which is hereby incorporated by reference in its entirety and for all purposes.

### REFERENCE TO A SEQUENCE LISTING, A TABLE OR A COMPUTER PROGRAM LISTING APPENDIX SUBMITTED AS AN ASCII TEXT FILE

The Sequence Listing written in file 048440-692001WO_SEQUENCE_LISTING_ST25.txt, created on Mar. 18, 2021, 23,377 bytes, machine format IBM-PC, MS Windows operating system, is hereby incorporated by reference.

### BACKGROUND

IL-15 is an important cytokine that activates T cells and NK cells and does not induce apoptosis. IL-15 binds to the IL-2/15β receptor and γC receptor. IL-15 also binds to the IL-15a receptor, a sushi domain protein that, in embodiments, interacts with IL-15 with 3.2 pM affinity and dramatically enhances the IL-15 affinity to IL-2/15β receptor and γC receptor. In order to improve the tissue specificity, IL-15 is typically fused to a targeting moiety (e.g., the C-terminus of an antibody). In addition, IL-15 has been fused to the sushi domain to improve its expression and affinity for the IL-2/IL-15β/γC receptor. A significant concern with this approach is on-target, off-tissue toxicities. Provided herein, inter alia, are solutions to this and other problems in the art.

### BRIEF SUMMARY OF THE INVENTION

In one aspect, a multivalent ligand binding complex is provided. The complex includes a first protein dimerizing domain non-covalently bound to a second protein dimerizing domain to form a first ligand binding domain, wherein the first protein dimerizing domain is covalently bound to a second ligand binding domain through a first chemical linker attached to the N-terminus of the first protein dimerizing domain. And the first protein dimerizing domain is covalently bound to a second ligand binding domain enhancer through a second chemical linker attached to the C-terminus of the first protein dimerizing domain.

In another aspect, a multivalent ligand binding complex is provided. The complex includes a first protein dimerizing domain non-covalently bound to a second protein dimerizing domain to form a first ligand binding domain, wherein the first protein dimerizing domain is covalently bound to a second ligand binding domain through a first chemical linker attached to the C-terminus of the first protein dimerizing domain. And the first protein dimerizing domain is covalently bound to a second ligand binding domain enhancer through a second chemical linker attached to the N-terminus of the first protein dimerizing domain.

In another aspect, a pharmaceutical composition is provided. The pharmaceutical composition includes a complex of any one of the previous embodiments and a pharmaceutically acceptable excipient.

### BRIEF DESCRIPTION OF THE DRAWINGS

FIGS. 1A-1D. The figures show two different configurations shown on one Fab chain. FIGS. 1A-1B show one possible configuration with sushi domain attached to the N-terminus of the light chain and IL-15 fused to the C-terminus of the light chain. FIGS. 1C-1D show a different possible configuration with sushi domain to the C-terminus of the light chain and IL-15 fused to the N-terminus of the light chain.

FIG. 2. The figure shows positions amenable to building disulfide bonds between sushi domain and IL-15, including residue 67 of sushi domain to residue 90 of IL-15 and residue 67 of sushi domain to residue 87 of IL-15.

FIG. 3. The figure shows that the positions for building disulfide bonds are remote from the receptor site.

FIG. 4. The figure provides a visual summary of second ligand binding domains and second ligand binding domain enhancers useful for the complexes and methods provided herein.

FIGS. 5A-5B. The figures are schematics showing the configurations of two different masked complexes and their activity in tumor microenvironments. FIG. 5A shows that the masked trastuzumab Fab-cytokine is inactive in normal tissues. Through antigen recognition the therapeutic is directed to the tumor site. At the tumor microenvironment, the complex is activated by tumor-specific proteases. In the example schematic, the sushi domain is attached to the N-terminus of the heavy chain and IL-2 is fused to the C-terminus of the heavy chain. FIG. 5B shows a different possible configuration of the trastuzumab IL-2 complex with the sushi domain attached to the C-terminus of the heavy chain and IL-15 fused to the N-terminus of the heavy chain.

FIGS. 6A-6D. The figures are representative images of non-reducing and reducing gels for analysis of non-cleaved and MMP-cleaved IL-2 Fab complexes. Prior to cleavage the complex has a molecular weight of approximately 65 kDa, the expected size of a Fab domain conjugated to IL2. Following cleavage, bands at either 50 kDa or 25 kDa are detected, which are representative of the Fab heavy chain and light chain, respectively. Gels are shown for FIG. 6A 1202-1014, FIG. 6B 1202-1015, FIG. 6C 1202-1016 and FIG. 6D 1202-1017 IL2-Fab complexes. As illustrated in samples with no MMP added, there is some non-MMP pre-cleavage of the complex.

FIGS. 7A-7C. The figures are representative images of gels showing cleavage products of the 1202-1015 IL2-Fab complex and cleavage sites of non-MMP proteases. FIG. 7A shows portions of the 1202-1015 IL2-Fab complex that were subject to non-MMP dependent cleavage. These cleavage products were verified by mass spectrometry to verify that they were cleaved by non-MMP proteases. FIG. 7B illustrates potential cleavage sites within or adjacent to the linker region of the IL2-meditope enabled Trastuzumab heavy chain IL2Ra complex (IL2_meTrasHC_IL2Rα, SEQ ID NO:9), as shown on the top panel. The arrows indicate where potential proteases (e.g. serine proteases, cathepsin G) recognize the EVQLVESG sequence (SEQ ID NO:11), and can cleave between the L and V residues. The sequence of the meditope enabled Trastuzumab light chain (meTraLC, SEQ ID NO:10) is illustrated on the bottom panel. FIG. 7C shows cleavage products of IL2-Fab complexes that were subject to mutations to avoid potential non-MMP dependent cleavage. Various mutations are as listed in Table 2.

FIG. 8. The figure shows a representative image of gel analysis of cleavage products for IL-15-Fab complexes 1215-1045, 1215-1046, 1215-1047 and 1215-1048. The 1215-1045 complex is activated by MMP, as illustrated by substantially complete cleavage reactions.

FIGS. 9A-9F. The figures illustrate surface plasmon resonance analysis detecting binding of IL-15-Fab complex to IL-15 receptor subunit β (IL-15R13)-Fc immobilized on an SPR chip. The data demonstrates that the complex binds to IL-15R13, even without the γ subunit. This indicates that IL-15R13 is accessible to the IL-15-Fab complex. Results are shown for FIG. 9A 1215-1045 Null, FIG. 9B 1215-1047 Null, FIG. 9C 1215-1045 MMP7, FIG. 9D 1215-1047, FIG. 9E 1215-1046 Null and FIG. 9F 1215-1048 Null IL2-Fab complexes. Binding constants for IL-15-Fab complex to IL-15 receptor subunit β (IL-15R13)-Fc are as shown in Table 5.

FIG. 10. The figure shows the melting temperature of various IL2-Fab complexes as measured by differential scanning fluorimetry. Results show that Cys mutations within the sushi domain or IL2 cytokine of the 1215-1047 and 1215-1048 complexes increased melting temperatures. The cysteine substitutions allow for disulfide bond formation between IL2 and the sushi domain. Melting temperatures are as shown in Table 6.

FIG. 11. The figure depicts data obtained by differential scanning fluorimetry used to determine the melting temperature of IL-15-sushi complexes including cysteine modifications as indicated (IL-15RaIL-15 mA including Cys67 and Cys87; IL-15RaIL-15mB including Cys67 and Cys90) and compared to the ‘parental’ molecule (IL-15-sushi complex with no cysteine modifications; IL-15 Superagonist; hFcIL-15RaIL-15). The 67/87 disulfide included in IL-15RaIL-15 mA (including Cys67 and Cys87) melted at a lower temperature (˜65° C.) than the parental molecule (IL-15 Superagonist; hFcIL-15RaIL-15) (˜80° C.). The 67/90 disulfide IL-15RaIL-15mB (including Cys67 and Cys90) melted at a higher temperature (˜82° C.).

### DETAILED DESCRIPTION

### Definitions

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

A “chemical linker,” as provided herein, is a covalent linker, a non-covalent linker, a peptide or peptidyl linker (a linker including a peptide moiety), a cleavable peptide linker, a substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene or any combination thereof. Thus, a chemical linker as provided herein may include a plurality of chemical moieties, wherein each of the plurality of chemical moieties is chemically different. Alternatively, the chemical linker may be a non-covalent linker. Examples of non-covalent linkers include without limitation, ionic bonds, hydrogen bonds, halogen bonds, van der Waals interactions (e.g. dipole-dipole, dipole-induced dipole, London dispersion), ring stacking (pi effects), and hydrophobic interactions. In embodiments, a chemical linker is formed using conjugate chemistry including, but not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition).

A “cleavable linker” refers to a linker including an element (e.g., peptide sequence) that is labile to cleavage upon suitable manipulation (e.g., protease activity). Accordingly, a cleavable linker may comprise any of a number of chemical entities, including amino acids, nucleic acids, or small molecules, among others. A cleavable linker may be cleaved by, for instance, chemical, enzymatic, or physical means. Non-limiting examples of labile elements included in cleavable linkers include protease cleavage sites, nucleic acid sequences cleaved by nucleases, photolabile, acid-labile, or base-labile functional groups. In embodiments, the chemical linker is a protease cleavable linker. In embodiments, the chemical linker is a tumor-associated protease cleavable linker.

In embodiments, the chemical linker includes a bovine serum albumin (BSA) binding moiety. In embodiments, the chemical linker is pH sensitive linker. The chemical linkers provided herein may include a BSA binding moiety (i.e., a peptide sequence capable of binding to BSA). At a physiological pH said BSA binding moiety is capable of binding to BSA. In embodiments, the BSA binding moiety does not bind to BSA at an acidic pH (e.g., a pH below 7, 6, 5, 4, 3, 2 or 1). While BSA binds to the BSA binding moiety at a physiological pH (i.e. neutral pH, pH 7), BSA increases the half-life and/or stability of the complex provided herein including embodiments thereof relative to the absence of BSA. Upon transition of the complex bound to BSA through the BSA binding moiety from a non-tumor environment to a tumor environment the pH may change from physiological to acidic thereby causing the BSA to dissociate from the BSA binding moiety and releasing the complex to bind to a cancer cell.

“Proteases” (or “proteinases”, “peptidases”, or “proteolytic” enzymes) generally refer to a class of enzymes that cleave peptide bonds between amino acids of proteins. Because proteases use a molecule of water to effect hydrolysis of peptide bonds, these enzymes can also be classified as hydrolases. Six classes of proteases are presently known: serine proteases, threonine proteases, cysteine proteases, aspartic acid proteases, metalloproteases, and glutamic acid proteases (see, e.g., Barrett A. J. et al. The Handbook of Proteolytic Enzymes, 2nd ed. Academic Press, 2003). A “tumor-associated protease” refers to a class of enzymes that cleave peptide bonds between amino acids of proteins, which is expressed in a tumor-environment (i.e., in and in proximity to the location of a tumor) by tumor and/or non-tumor cells.

Proteases are involved in a multitude of physiological reactions from simple digestion of food proteins to highly regulated cascades (e.g., the cell cycle, the blood clotting cascade, the complement system, and apoptosis pathways). It is well known to the skilled artisan that proteases can break either specific peptide bonds, depending on the amino acid sequence of a protein, or break down a polypeptide to constituent amino acids.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different the general chemical structure of a nucleic acid, but that functions in a manner similar to a naturally occurring nucleic acid. Examples of such analogues include, without limitation, phosphorothioates, phosphoramidates, methyl phosphonates, chiral-methyl phosphonates, 2-O-methyl ribonucleotides, and peptide-nucleic acids (PNAs).

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., IL-15) in other proteins with different numbering systems. For example, by performing a simple sequence alignment with a protein (e.g., IL-15) the identity and location of residues corresponding to specific positions of the protein are identified in other protein sequences aligning to the protein. For example, a selected residue in a selected protein corresponds to glutamic acid at position 90 when the selected residue occupies the same essential spatial or other structural relationship as a glutamic acid at position 90. In some embodiments, where a selected protein is aligned for maximum homology with a protein, the position in the aligned selected protein aligning with glutamic acid 90 is the to correspond to glutamic acid 90. Instead of a primary sequence alignment, a three dimensional structural alignment can also be used, e.g., where the structure of the selected protein is aligned for maximum correspondence with the glutamic acid at position 90, and the overall structures compared. In this case, an amino acid that occupies the same essential position as glutamic acid 90 in the structural model is to correspond to the glutamic acid 90 residue.

Likewise, a selected residue in a selected protein or protein domain (e.g., a second ligand binding domain or a second ligand binding domain enhancer) corresponds to a residue at position 67, when the selected residue occupies the same essential spatial or other structural position within the protein or protein domain as the residue at position 67. In some embodiments, where a selected protein or protein domain is aligned for maximum homology with, the position in the aligned selected protein or protein domain (e.g., a second ligand binding domain or a second ligand binding domain enhancer) aligning with position 67 is said to correspond to position 67. Instead of a primary sequence alignment, a three dimensional structural alignment can also be used, e.g., where the structure of the selected protein or protein domain is aligned for maximum correspondence with the residue at position 67, and the overall structures compared. In this case, an amino acid that occupies the same essential position as residue 67 in the structural model is said to correspond to the 67 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“Interleukin 15” or “IL-15” or as referred to herein includes any of the recombinant or naturally-occurring forms of IL-15 protein or variants or homologs thereof that maintain IL-15 activity (e.g. within at least 50%, 80%, 90%, 95%, 96%, 97%, 98%, 99% or 100% activity compared to IL-15 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-15 protein. In embodiments, the IL-15 protein is substantially identical to the protein identified by the NCBI Reference Sequence: NP_001230468.1 or a variant or homolog having substantial identity thereto.

“IL-15RA” as referred to herein includes any of the recombinant or naturally-occurring forms of IL-15 receptor alpha protein or variants or homologs thereof that maintain IL-15 receptor alpha activity (e.g. within at least 50%, 80%, 90%, 95%, 96%, 97%, 98%, 99% or 100% activity compared to IL-15 receptor alpha protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-15 receptor alpha protein. In embodiments, the IL-15 receptor alpha protein is substantially identical to the protein identified by the NCBI Reference Sequence: NP_000576.1 or a variant or homolog having substantial identity thereto. In embodiments, the IL-15 receptor alpha protein is substantially identical to the protein identified by the NCBI Reference Sequence: NP_001243694.1 or a variant or homolog having substantial identity thereto. In embodiments, the IL-15 receptor alpha protein is substantially identical to the protein identified by the NCBI Reference Sequence: NP_002180.1 or a variant or homolog having substantial identity thereto. In embodiments, the IL-15 receptor alpha protein is substantially identical to the protein identified by the NCBI Reference Sequence: NP_751950.2 or a variant or homolog having substantial identity thereto. In embodiments, the IL-15 receptor alpha protein is substantially identical to the protein identified by the NCBI Reference Sequence: NP_001338024.1 or a variant or homolog having substantial identity thereto. In embodiments, the second ligand binding domain enhancer is an IL-15 domain enhancer. In embodiments, the IL-15 domain enhancer is an IL-15RA domain. In embodiments, the IL-15RA domain includes a sushi domain. In embodiments, the IL-15RA domain is a sushi domain.

“Interleukin 2” or “IL-2” or as referred to herein includes any of the recombinant or naturally-occurring forms of IL-2 protein or variants or homologs thereof that maintain IL-2 activity (e.g. within at least 50%, 80%, 90%, 95%, 96%, 97%, 98%, 99% or 100% activity compared to IL-2 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-2 protein. In embodiments, the IL-2 protein is substantially identical to the protein identified by the UniProt reference number: P60568 or a variant or homolog having substantial identity thereto.

“IL-2RA” as referred to herein includes any of the recombinant or naturally-occurring forms of IL-2 receptor alpha protein or variants or homologs thereof that maintain IL-2 receptor alpha activity (e.g. within at least 50%, 80%, 90%, 95%, 96%, 97%, 98%, 99% or 100% activity compared to IL-2 receptor alpha protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-2 receptor alpha protein. In embodiments, the IL-2 receptor alpha protein is substantially identical to the protein identified by the UniProt reference number: P01589.

“Carcinoembryonic antigen” (CEA) as referred to herein describes a set of highly related glycoproteins involved in cell adhesion and includes any of the recombinant or naturally-occurring forms of CEA variants or homologs thereof that maintain CEA activity (e.g. within at least 50%, 80%, 90%, 95%, 96%, 97%, 98%, 99% or 100% activity compared to CEA). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to naturally occurring CEA proteins.

An “PD-1 protein” or “PD-1” as referred to herein includes any of the recombinant or naturally-occurring forms of the Programmed cell death protein 1 (PD-1) also known as cluster of differentiation 279 (CD 279) or variants or homologs thereof that maintain PD-1 protein activity (e.g. within at least 50%, 80%, 90%, 95%, 96%, 97%, 98%, 99% or 100% activity compared to PD-1 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-1 protein. In embodiments, the PD-1 protein is substantially identical to the protein identified by the UniProt reference number Q15116 or a variant or homolog having substantial identity thereto. In embodiments, the PD-1 protein is substantially identical to the protein identified by the UniProt reference number Q02242 or a variant or homolog having substantial identity thereto.

An “PD-L1 protein” or “PD-L1” as referred to herein includes any of the recombinant or naturally-occurring forms of the Programmed death ligand 1 (PD-L1) also known as cluster of differentiation 274 (CD 274) or B7 homolog, or variants or homologs thereof that maintain PD-L1 protein activity (e.g. within at least 50%, 80%, 90%, 95%, 96%, 97%, 98%, 99% or 100% activity compared to PD-L1 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-L1 protein. In embodiments, the PD-L1 protein is substantially identical to the protein identified by the UniProt reference number Q9NZQ7 or a variant or homolog having substantial identity thereto. In embodiments, the PD-L1 protein is substantially identical to the protein identified by the UniProt reference number Q9EP73 or a variant or homolog having substantial identity thereto.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab2, bispecific Fab2, trispecific Fab3, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR H2”, “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The term “antigen” as provided herein refers to molecules capable of binding to the antibody binding domain provided herein. An “antigen binding domain” as provided herein is a region of an antibody that binds to an antigen (epitope). As described above, the antigen binding domain is generally composed of one constant and one variable domain of each of the heavy and the light chain (VL, VH, CL and CH1, respectively). The paratope or antigen-binding site is formed on the N-terminus of the antigen binding domain. The two variable domains of an antigen binding domain typically bind the epitope on an antigen.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a ligand binding domain provided herein. A “ligand binding domain” refers to a molecule capable of binding a receptor, a soluble molecule, an antibody, antibody variant, antibody region or fragment thereof. Therefore a ligand may be a protein expressed on the surface of a cell, for example a membrane-bound receptor (e.g., an interleukin receptor) or an antigen expressed on the surface of a cell (e.g., a cancer antigen). The term “ligand” and “target ligand” as used herein can be used interchangeably.

Contacting” is used in accordance with its plain ordinary meaning and refers to the process of allowing at least two distinct species (e.g. chemical compounds including biomolecules or cells) to become sufficiently proximal to react, interact or physically touch. It should be appreciated, that the resulting reaction product can be produced directly from a reaction between the added reagents or from an intermediate from one or more of the added reagents which can be produced in the reaction mixture.

The term “contacting” may include allowing two species to react, interact, or physically touch (e.g., bind), wherein the two species may be, for example, an antibody construct as described herein and a cancer protein. In embodiments, contacting includes, for example, allowing an antibody construct to bind to a cancer protein expressed on a cancer cell.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The term “plasmid,” “expression vector,” or “viral vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids. Suitable viral vectors contemplated herein include, for example, lentiviral vectors and onco-retroviral vectors.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In some embodiments, the sample is obtained from a human.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma (Mantel cell lymphoma), head and neck cancer, colorectal cancer, prostate cancer, pancreatic cancer, melanoma, breast cancer, neuroblastoma).

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P388 leukemia model is widely accepted as being predictive of in vivo anti-leukemic activity. It is believed that a compound that tests positive in the P388 assay will generally exhibit some level of anti-leukemic activity in vivo regardless of the type of leukemia being treated. Accordingly, the present application includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “associated” or “associated with” in the context of a substance or substance activity or function associated with a disease (e.g., cancer) means that the disease is caused by (in whole or in part), or a symptom of the disease is caused by (in whole or in part) the substance or substance activity or function.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. For example, certain methods herein treat cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma). For example, certain methods herein treat cancer by decreasing or reducing or preventing the occurrence, growth, metastasis, or progression of cancer; or treat cancer by decreasing a symptom of cancer. Symptoms of cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) would be known or may be determined by a person of ordinary skill in the art.

As used herein the terms “treatment,” “treat,” or “treating” refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

An “effective amount” is an amount sufficient to accomplish a stated purpose (e.g. achieve the effect for which it is administered, treat a disease, reduce enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

### Complexes

Provided herein are, inter alia, multivalent complexes capable of binding more than one antigen (ligand). The complexes provided herein may include a first ligand binding domain (e.g., a Fab) capable of binding a tumor antigen. The complexes further include a second ligand binding domain (e.g., IL-15) non-covalently and/or covalently attached to a second ligand binding domain enhancer. The second ligand binding domain enhancer can increase the stability of the second ligand binding domain (e.g., IL-15, IL-2) and its affinity to its innate receptor. The second ligand binding domain enhancer may also reduce the entropy of the second ligand binding domain (e.g., IL-15, IL-2) relative to the absence of the second ligand binding domain (e.g., IL-15, IL-2). The second ligand binding domain enhancer may be an IL-15RA receptor domain or an IL-2RA receptor domain. In embodiments, the second ligand binding domain enhancer is a sushi domain (an extracellular domain of IL-15RA or IL-2RA).

As defined herein, the term “enhancing”, “enhancer”, and the like in reference to a ligand binding domain enhancer provided herein means positively affecting the biological function (e.g., by increasing binding, or targeted delivery) of the ligand binding domain, which the enhancer binds to. In some embodiments, enhancing refers to the ability to increase the binding affinity or the structural stability of the ligand binding domain relative to the absence of the enhancer. In some embodiments, enhancing refers to increasing the target specificity and the targeted delivery of the ligand binding domain relative to the absence of the enhancer. In some embodiments, enhancing refers to the decrease of unspecific binding of the ligand binding domain relative to the absence of the enhancer. Thus, enhancing includes, at least in part, partially or totally increasing activity, target specificity, or binding affinity of a ligand binding domain relative to the absence of the ligand binding domain enhancer. In embodiments, the multivalent or covalent complexes provided herein are administered to a subject in need for therapeutic treatment (e.g., cancer treatment) and the ligand binding domain enhancer increases the targeted delivery of the ligand binding domain relative to the absence of ligand binding domain enhancer. In some embodiments, the first or second chemical linker are cleaved by a cancer-specific protease resulting in the release of the ligand binding domain (e.g., IL-2 domain) from the multivalent complex and subsequent binding of the ligand binding domain to its target ligand (e.g., IL-2 receptor).

In embodiments, the ligand binding domain (IL-15) binds the target ligand while bound to the ligand binding domain enhancer (sushi domain). In further embodiments, the binding of the ligand binding domain to the target ligand is increased relative to the absence of the ligand binding domain enhancer.

As defined herein, the term “enhancing”, “increasing” and the like in reference to a ligand binding domain-ligand binding domain enhancer interaction means positively affecting (e.g. increasing) the activity or function of the ligand binding domain (e.g. IL-15, IL-2) relative to the activity or function of the ligand biding domain in the absence of the enhancer. In embodiments, enhancing refers to an increase of activity (binding activity, binding affinity) of the ligand binding domain resulting from a direct interaction (e.g. an enhancer binds to the ligand binding domain).

The enhancer can increase activity, stability or binding affinity 10%, 20%, 30%, 40%, 50%, 60%, 70%, 80%, 90% or more in comparison to a control in the absence of the enhancer. In certain instances, activity, stability or binding affinity is 1.5-fold, 2-fold, 3-fold, 4-fold, 5-fold, 10-fold or higher than the activity, stability or binding affinity in the absence of the enhancer.

The complexes provided herein including embodiments thereof provide, inter alia, therapeutic means to deliver cancer-specific antibodies (first ligand binding domain) to the cancer site and at the same time activate effector cells in the tumor environment through cytokine-dependent cell proliferation, antibody-dependent cellular cytotoxicity (ADCC) or direct cell killing mediated by the second ligand domain including, for example, IL-15 and IL-15Ra.

The term “multivalent ligand binding complex” as provided herein refers to a multivalent polypeptide complex including at least two binding domains, wherein each of the binding domains binds to a ligand. The ligand binding domains included in the multivalent ligand binding complex may be bound to each other non-covalently or covalently through chemical linkers.

In one aspect, a multivalent ligand binding complex is provided. The complex includes a first protein dimerizing domain non-covalently bound to a second protein dimerizing domain to form a first ligand binding domain, where the first protein dimerizing domain is covalently bound to a second ligand binding domain through a first chemical linker attached to the N-terminus of the first protein dimerizing domain. And the first protein dimerizing domain is covalently bound to a second ligand binding domain enhancer through a second chemical linker attached to the C-terminus of the first protein dimerizing domain.

In another aspect, a multivalent ligand binding complex is provided. The complex includes a first protein dimerizing domain non-covalently bound to a second protein dimerizing domain to form a first ligand binding domain, where the first protein dimerizing domain is covalently bound to a second ligand binding domain through a first chemical linker attached to the C-terminus of the first protein dimerizing domain. And the first protein dimerizing domain is covalently bound to a second ligand binding domain enhancer through a second chemical linker attached to the N-terminus of the first protein dimerizing domain.

A “ligand binding domain” as provided herein refers to a peptide domain capable of selectively binding to a target ligand. A ligand binding domain may covalently or non-covalently bind to a target ligand. Non-limiting examples of ligand binding domains include single chain antibodies, chemokines (e.g., interleukins), antibody variants or fragments thereof, antibodies or fragments thereof. In embodiments, the ligand binding domain is a Fab. In embodiments, the ligand binding domain is a chemokine, variant or fragment thereof.

In embodiments, the second ligand binding domain is an IL-15 domain. An IL-15 domain as provided herein refers to a protein domain including any of the recombinant or naturally-occurring forms of IL-15 protein, functional fragments (shorter than naturally or recombinant occurring forms of IL-15), or variants or homologs thereof that maintain IL-15 activity (e.g. within at least 50%, 80%, 90%, 95%, 96%, 97%, 98%, 99% or 100% activity compared to IL-15 protein). In some aspects, the variants, functional fragments or homologs forming an IL-15 domain have at least 90%, 95%, 96%, 97%, 98%, 99% or 100% amino acid sequence identity across the whole sequence or in case of a fragment, a portion of the sequence (e.g. a 50, 100, 150 or 200 continuous amino acid portion) compared to a naturally occurring IL-15 protein.

In embodiments, the second ligand binding domain is an IL-2 domain. An IL-2 domain as provided herein refers to a protein domain including any of the recombinant or naturally-occurring forms of IL-2 protein, functional fragments (proteins that are shorter than naturally or recombinant occurring forms of IL-2) or variants or homologs thereof, that maintain IL-2 activity (e.g. within at least 50%, 80%, 90%, 95%, 96%, 97%, 98%, 99% or 100% activity compared to IL-2 protein). In some aspects, the variants, functional fragments or homologs forming an IL-2 domain have at least 90%, 95%, 96%, 97%, 98%, 99% or 100% amino acid sequence identity across the whole sequence or in case of a functional fragment, a portion of the sequence (e.g. a 50, 100, 150 or 200 continuous amino acid portion) compared to a naturally occurring IL-2 protein.

The ligand binding domains provided herein (e.g., first, second binding domain) may bind a plurality (at least two, e.g., 2, 3, 4) of the same type of ligand, two or more different regions of the same ligand or a plurality of (two or more) different ligands. Wherein the ligand binding domains provided herein bind a plurality of the same type of ligand, the same type of ligand may form part of one cell or of two or more different cells and the ligand binding domains bind separate ligand molecules. Alternatively, the ligand binding domains provided herein may bind two or more different regions of the same ligand (e.g., different epitopes of the same protein). Further, the ligand binding domains provided herein may bind a plurality of different ligands and the plurality of different ligands may form part of one cell or a plurality of cells.

In embodiments, the first ligand binding domain is different from the second ligand binding domain. The first ligand binding domain may be a protein domain including two protein dimerizing domains (e.g., a first and a second protein dimerizing domain). The first protein dimerizing domain and the second protein dimerizing domain may be covalently and/or non-covalently bound to each other. Thus, in embodiments, the first protein dimerizing domain is bound to the second protein dimerizing domain. In embodiments, the peptide further includes a covalent bond connecting the first protein dimerizing domain and the second protein dimerizing domain. In embodiments, the covalent bond is a disulfide bond. In embodiments, the first protein dimerizing domain is a Fab domain.

In embodiments, the second ligand binding domain is an interleukin domain. In embodiments, the second ligand binding domain is non-covalently bound to the second ligand binding domain enhancer. In embodiments, the second ligand binding domain is covalently bound to the second ligand binding domain enhancer through one or more disulfide linkages. In embodiments, the second ligand binding domain includes a cysteine at a position corresponding to position 45, 87 or 90 of the second ligand binding domain. In embodiments, the second ligand binding domain includes a cysteine at a position corresponding to position 45 of the second ligand binding domain. In embodiments, the second ligand binding domain includes a cysteine at a position corresponding to position 87 of the second ligand binding domain. In embodiments, the second ligand binding domain includes a cysteine at a position corresponding to position 90 of the second ligand binding domain.

In embodiments, the second ligand binding domain is an interleukin domain. In embodiments, the second ligand binding domain is an IL-15 domain. In embodiments, the IL-15 domain is non-covalently bound to the second ligand binding domain enhancer. In embodiments, the IL-15 domain is covalently bound to the second ligand binding domain enhancer through one or more disulfide linkages. In embodiments, the IL-15 domain includes a cysteine at a position corresponding to position 45, 87 or 90 of the IL-15 domain. In embodiments, the IL-15 domain includes a cysteine at a position corresponding to position 45 of the IL-15 domain. In embodiments, the IL-15 domain includes a cysteine at a position corresponding to position 87 of the IL-15 domain. In embodiments, the IL-15 domain includes a cysteine at a position corresponding to position 90 of the IL-15 domain.

In embodiments, the second ligand binding domain enhancer includes a cysteine at a position corresponding to position 37, 38, 68 or 67 of the second ligand binding domain enhancer. In embodiments, the second ligand binding domain enhancer includes a cysteine at a position corresponding to position 37 of the second ligand binding domain enhancer. In embodiments, the second ligand binding domain enhancer includes a cysteine at a position corresponding to position 38 of the second ligand binding domain enhancer. In embodiments, the second ligand binding domain enhancer includes a cysteine at a position corresponding to position 68 of the second ligand binding domain enhancer. In embodiments, the second ligand binding domain enhancer includes a cysteine at a position corresponding to position 67 of the second ligand binding domain enhancer.

In embodiments, the second ligand binding domain enhancer is an IL-15 domain enhancer. In embodiments, the IL-15 domain enhancer includes a cysteine at a position corresponding to position 37, 38, 68 or 67 of the IL-15 domain enhancer. In embodiments, the IL-15 domain enhancer includes a cysteine at a position corresponding to position 37 of the IL-15 domain enhancer. In embodiments, the IL-15 domain enhancer includes a cysteine at a position corresponding to position 38 of the IL-15 domain enhancer. In embodiments, the IL-15 domain enhancer includes a cysteine at a position corresponding to position 68 of the IL-15 domain enhancer. In embodiments, the IL-15 domain enhancer includes a cysteine at a position corresponding to position 67 of the IL-15 domain enhancer.

In embodiments, the first ligand binding domain is different from the second ligand binding domain. In embodiments, the complex further includes a covalent bond connecting the first protein dimerizing domain and the second protein dimerizing domain. In embodiments, the first protein dimerizing domain is bound to the second protein dimerizing domain.

In embodiments, the first chemical linker is bound to the C-terminus of the second ligand binding domain and the second chemical linker is bound to the N-terminus of the second ligand binding domain enhancer. In embodiments, the first chemical linker is bound to the C-terminus of the second ligand binding domain enhancer and the second chemical linker is bound to the N-terminus of the second ligand binding domain.

In embodiments, the first protein dimerizing domain includes a variable light chain domain. In embodiments, the first protein dimerizing domain includes a constant light chain domain.

A “light chain variable (VL) domain” as provided herein refers to the variable region of the light chain of an antibody, an antibody variant or fragment thereof. Likewise, the “heavy chain variable (VH) domain” as provided herein refers to the variable region of the heavy chain of an antibody, an antibody variant or fragment thereof. As described above, the light chain variable domain and the heavy chain variable domain together form the paratope, which binds an antigen (epitope). The paratope or antigen-binding site is formed at the N-terminus of an antibody, an antibody variant or fragment thereof. In embodiments, the light chain variable (VL) domain includes CDR L1, CDR L2, CDR L3 and FR L1, FR L2, FR L3 and FR L4 (framework regions) of an antibody light chain. In embodiments, the heavy chain variable (VH) domain includes CDR H1, CDR H2, CDR H3 and FR H1, FR H2, FR H3 and FR H4 (framework regions) of an antibody heavy chain. In embodiments, the light chain variable (VL) domain and a light chain constant (CL) domain form part of an antibody light chain. In embodiments, the heavy chain variable (VH) domain and a heavy chain constant (CH1) domain form part of an antibody heavy chain. In embodiments, the heavy chain variable (VH) domain and one or more heavy chain constant (CH1, CH2, or CH3) domains form part of an antibody heavy chain. In embodiments, the light chain variable (VL) domain forms part of an antibody fragment. In embodiments, the heavy chain variable (VH) domain forms part of an antibody fragment. In embodiments, the light chain variable (VL) domain forms part of an antibody variant. In embodiments, the heavy chain variable (VH) domain forms part of an antibody variant. In embodiments, the light chain variable (VL) domain forms part of a Fab. In embodiments, the heavy chain variable (VH) domain forms part of a Fab. In embodiments, the light chain variable (VL) domain forms part of a scFv. In embodiments, the heavy chain variable (VH) domain forms part of a scFv.

In embodiments, the constant light chain domain is bound to the second ligand binding domain through the variable light chain domain. In embodiments, the constant light chain domain is bound to the second ligand binding domain enhancer through the variable light chain domain. In embodiments, the first protein dimerizing domain is an antibody light chain.

In embodiments, the first protein dimerizing domain includes a variable heavy chain domain. In embodiments, the first protein dimerizing domain includes a constant heavy chain domain. In embodiments, the constant heavy chain domain is bound to the second ligand binding domain through the variable heavy chain domain. In embodiments, the constant heavy chain domain is bound to the second ligand binding domain enhancer through the variable heavy chain domain. In embodiments, the first protein dimerizing domain is an antibody heavy chain.

In embodiments, the second protein dimerizing domain includes a constant heavy chain domain. In embodiments, the second protein dimerizing domain includes a variable heavy chain domain. In embodiments, the second protein dimerizing domain is an antibody heavy chain. In embodiments, the second protein dimerizing domain includes a constant light chain domain. In embodiments, the second protein dimerizing domain includes a variable light chain domain. In embodiments, the second protein dimerizing domain is an antibody light chain.

In embodiments, the first ligand binding domain is a Fab domain. In embodiments, the first protein dimerizing domain is bound to an Fc domain through a third chemical linker. In embodiments, the second protein dimerizing domain is bound to an Fc domain through a third chemical linker. In embodiments, the first ligand binding domain is an anti PDL-1 binding domain, an anti L1CAM binding domain, an anti-EGFR binding domain or an anti-CEA binding domain. In embodiments, the first ligand binding domain is an anti-Her2 binding domain, an anti-Her3 binding domain, an anti-Her4 binding domain, an anti-cMet binding domain, an anti-IGFR binding domain, an anti-CDH6 binding domain, an anti-Ax1 binding domain, an anti-Tissue factor binding domain, an anti-Mesothelin binding domain or a binding domain that binds to MHC loaded with tumor peptides. In embodiments, the first ligand binding domain is an anti PDL-1 binding domain. In embodiments, the first ligand binding domain is an anti L1CAM binding domain. In embodiments, the first ligand binding domain is an anti-EGFR binding domain. In embodiments, the first ligand binding domain is an anti-CEA binding domain. In embodiments, the first ligand binding domain is an anti-Her2 binding domain. In embodiments, the first ligand binding domain is an anti-Her3 binding domain. In embodiments, the first ligand binding domain is an anti-Her4 binding domain. In embodiments, the first ligand binding domain is an anti-cMet binding domain. In embodiments, the first ligand binding domain is an anti-IGFR binding domain. In embodiments, the first ligand binding domain is an anti-CDH6 binding domain. In embodiments, the first ligand binding domain is an anti-Ax1 binding domain. In embodiments, the first ligand binding domain is an anti-Tissue factor binding domain. In embodiments, the first ligand binding domain is an anti-Mesothelin binding domain. In embodiments, the first ligand binding domain is a binding domain that binds to WIC loaded with tumor peptides.

Wherein the first ligand binding domain is an antibody, variant or fragment thereof, it may be covalently or non-covalently attached to a peptide compound. The peptide compound provided herein may include a peptidyl moiety also referred to herein as “meditope.” Any of the meditopes and meditope-antibody complexes described in WO 2013/055404 or WO 2019/028190, which are incorporated herein in their entirety and for all purposes, may be used for the compositions or methods provided herein. The modified antibodies as described herein, including embodiments thereof, may be referred to herein, for example in the Examples, as meditope-enabled (me) antibodies. In embodiments, the meditope-enabled antibody is a monoclonal antibody (memAb). In embodiments, the meditope-enabled antibody is a humanized antibody. In embodiments, the Fab region of an antibody may be meditope enabled. In embodiments, the meditope-enabled antibody is a Fab. The term “meditope” as used herein refers to a peptidyl moiety included in the peptide compound as described herein. Thus, in embodiments, a meditope is a peptidyl moiety.

Meditope-enabled antibodies allow for the binding (e.g., covalent, non-covalent) of peptidyl moieties to a region in the Fab portion of the antibody without negatively influencing antibody binding site behavior. The peptidyl moieties (also referred to herein as meditopes) may be functionalized. For example, the peptidyl moieties may be conjugated to therapeutic or diagnostic agents through a covalent linker (e.g., using, for example, suitable reactive groups and click chemistry). A functionalized peptidyl moiety may be referred to herein as a peptide compound. The ability of the antibody to bind (covalently, non-covalently) a peptide compound endows the meditope-enabled antibody with the functionality to simultaneously target its specific antigen via its antibody binding site and deliver a therapeutic or diagnostic agent.

The term “peptidyl” and “peptidyl moiety” refers to a peptide attached to the remainder of a molecule. A peptidyl moiety may be substituted with a chemical linker that serves to attach the peptidyl moiety to a molecule. The peptidyl moiety may also be substituted with additional chemical moieties (e.g., additional R substituents). The term “meditope” as used herein refers to a peptidyl moiety included in the peptide compound as described herein. Thus, in embodiments, a meditope is a peptidyl moiety.

The peptidyl moiety (e.g., meditope) may be a linear or a cyclic peptide moiety. Various methods for cyclization of a peptide moiety may be used, e.g., to address in vivo stability and to enable chemo-selective control for subsequent conjugation chemistry. In some embodiments, the cyclization strategy is a lactam cyclization strategy, including head-to-tail (head-tail) lactam cyclization (between the terminal residues of the acyclic peptide) and/or lactam linkage between other residues. Lactam formation may also be affected by incorporating residues such as glycine, 0-Ala, and/or 7-aminoheptanoic acid, and the like, into the acyclic peptide cyclization precursors to produce different lactam ring sizes and modes of connectivity. Additional cyclization strategies such as “click” chemistry and olefin metathesis also can be used. Such methods of peptide and peptidomimetic cyclization are well known in the art. In embodiments, the peptidyl moiety (e.g., meditope) is a linear peptidyl moiety (e.g., linear meditope). In embodiments, the peptidyl moiety (e.g., meditope) is a cyclic peptidyl moiety (e.g., cyclic meditope).

The term “peptide compound” refers to a compound including a peptidyl portion. In embodiments, the peptide compound includes a peptide or peptidyl moiety directly (covalently) or indirectly (non-covalently) attached to one or more chemical substituents. In embodiments, the peptide compound includes a peptidyl moiety. In embodiments, the peptide compound is a compound as described in WO 2013/055404 or WO 2019/028190. Thus, the complexes provided herein may include a non-covalent linker including a peptidyl moiety, wherein the peptidyl moiety is a meditope. In embodiments, the chemical linker is a non-covalent peptidyl linker including a meditope. In embodiments, the chemical linker is a covalent peptidyl linker including a meditope.

In embodiments, the second ligand binding domain is a chemokine domain. In embodiments, the second ligand binding domain is an interleukin domain. In embodiments, the second ligand binding domain is an IL-2 domain, an IL-4 domain, an IL-7 domain, an IL-9 domain, an IL-15 domain, an IL-21 domain or a thymic stromal lymphopoietin (TSLP) domain. In embodiments, the second ligand binding domain enhancer is a chemokine domain enhancer. In embodiments, the second ligand binding domain enhancer is an interleukin domain enhancer. In embodiments, the second ligand binding domain enhancer includes a sushi domain. In embodiments, the second ligand binding domain enhancer is an IL-2 domain enhancer, an IL-4 domain enhancer, an IL-7 domain enhancer, an IL-9 domain enhancer, an IL-15 domain enhancer, an IL-21 domain enhancer or a thymic stromal lymphopoietin (TSLP) domain enhancer.

In embodiments, the first chemical linker is a peptidyl linker. In embodiments, the second chemical linker is a peptidyl linker. In embodiments, the first chemical linker and the second chemical linker are independently a covalent linker or a non-covalent linker. In embodiments, the first chemical linker and the second chemical linker are independently a cleavable peptide linker. In embodiments, the first chemical linker and the second chemical linker are independently an enzymatically cleavable linker. In embodiments, the first chemical linker and the second chemical linker are independently a protease cleavable linker. In embodiments, the first chemical linker and the second chemical linker are independently a tumor-associated protease cleavable linker. In embodiments, the first chemical linker and the second chemical linker independently have a length of about 0 to about 15 amino acid residues. In embodiments, the first chemical linker and the second chemical linker independently comprise a BSA binding moiety.

In embodiments, the first chemical linker, the second chemical linker and the third chemical linker are independently a cleavable peptide linker, including a protease cleavage site. A “cleavage site” as used herein, refers to a recognizable site for cleavage of a portion of a linker described herein. Thus, a cleavage site may be found in the sequence of a cleavable peptide linker as described herein, including embodiments thereof. In embodiments, the cleavage site is an amino acid sequence that is recognized and cleaved by a cleaving agent (e.g., a peptidyl sequence). In embodiments, the protease cleavage site includes the sequence of SEQ ID NO:11. In embodiments, the protease cleavage site is the sequence of SEQ ID NO:11. Exemplary cleaving agents include proteins, enzymes, DNAzymes, RNAzymes, metals, acids, and bases. In embodiments, the protease cleavage site is a tumor-associated protease cleavage site. A “tumor-associated protease cleavage site” as provided herein is an amino acid sequence recognized by a protease, whose expression is specific for a tumor cell or tumor cell environment thereof. In embodiments, the protease cleavage site is a matrix metalloprotease (MMP) cleavage site, a disintegrin and metalloprotease domain-containing (ADAM) metalloprotease cleavage site, a prostate specific antigen (PSA) protease cleavage site, a urokinase-type plasminogen activator (uPA) protease cleavage site, a membrane type serine protease 1 (MT-SP1) protease cleavage site or a legumain protease cleavage site. In embodiments, the matrix metalloprotease (MMP) cleavage site is a MMP 9 cleavage site, a MMP 13 cleavage site or a MMP 2 cleavage site. In embodiments, the disintegrin and metalloprotease domain-containing (ADAM) metalloprotease cleavage site is a ADAM 9 metalloprotease cleavage site, a ADAM 10 metalloprotease cleavage site or a ADAM 17 metalloprotease cleavage site.

In embodiments, the first chemical linker includes the sequence of SEQ ID NO:1. In embodiments, the first chemical linker includes the sequence of SEQ ID NO:3. In embodiments, the first chemical linker includes the sequence of SEQ ID NO:5. In embodiments, the first chemical linker includes the sequence of SEQ ID NO:7. In embodiments, the first chemical linker includes the sequence of SEQ ID NO:17. In embodiments, the first chemical linker includes the sequence of SEQ ID NO:19. In embodiments, the first chemical linker includes the sequence of SEQ ID NO:21. In embodiments, the first chemical linker includes the sequence of SEQ ID NO:23. In embodiments, the first chemical linker includes the sequence of SEQ ID NO:25. In embodiments, the first chemical linker includes the sequence of SEQ ID NO:27. In embodiments, the first chemical linker includes the sequence of SEQ ID NO:29. In embodiments, the first chemical linker includes the sequence of SEQ ID NO:31. In embodiments, the first chemical linker includes the sequence of SEQ ID NO:33. In embodiments, the first chemical linker includes the sequence of SEQ ID NO:35. In embodiments, the first chemical linker includes the sequence of SEQ ID NO:37.

In embodiments, the first chemical linker includes a sequence that has at least 90%, 95%, 96%, 97%, 98%, 99% or 100% amino acid sequence identity across the whole sequence or a portion of the sequence (e.g. a 10, 50, 100 continuous amino acid portion) of SEQ ID NO:1, SEQ ID NO:3, SEQ ID NO:5, SEQ ID NO:7, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, or SEQ ID NO:37.

In embodiments, the first chemical linker is the sequence of SEQ ID NO:1. In embodiments, the first chemical linker is the sequence of SEQ ID NO:3. In embodiments, the first chemical linker is the sequence of SEQ ID NO:5. In embodiments, the first chemical linker is the sequence of SEQ ID NO:7. In embodiments, the first chemical linker is the sequence of SEQ ID NO:17. In embodiments, the first chemical linker is the sequence of SEQ ID NO:19. In embodiments, the first chemical linker is the sequence of SEQ ID NO:21. In embodiments, the first chemical linker is the sequence of SEQ ID NO:23. In embodiments, the first chemical linker is the sequence of SEQ ID NO:25. In embodiments, the first chemical linker is the sequence of SEQ ID NO:27. In embodiments, the first chemical linker is the sequence of SEQ ID NO:29. In embodiments, the first chemical linker is the sequence of SEQ ID NO:31. In embodiments, the first chemical linker is the sequence of SEQ ID NO:33. In embodiments, the first chemical linker is the sequence of SEQ ID NO:35. In embodiments, the first chemical linker is the sequence of SEQ ID NO:37.

In embodiments, the second chemical linker includes the sequence of SEQ ID NO:2. In embodiments, the second chemical linker includes the sequence of SEQ ID NO:4. In embodiments, the second chemical linker includes the sequence of SEQ ID NO:6. In embodiments, the second chemical linker includes the sequence of SEQ ID NO:8. In embodiments, the second chemical linker includes the sequence of SEQ ID NO:18. In embodiments, the second chemical linker includes the sequence of SEQ ID NO:20. In embodiments, the second chemical linker includes the sequence of SEQ ID NO:22. In embodiments, the second chemical linker includes the sequence of SEQ ID NO:24. In embodiments, the second chemical linker includes the sequence of SEQ ID NO:26. In embodiments, the second chemical linker includes the sequence of SEQ ID NO:28. In embodiments, the second chemical linker includes the sequence of SEQ ID NO:30. In embodiments, the second chemical linker includes the sequence of SEQ ID NO:32. In embodiments, the second chemical linker includes the sequence of SEQ ID NO:34. In embodiments, the second chemical linker includes the sequence of SEQ ID NO:36. In embodiments, the second chemical linker includes the sequence of SEQ ID NO:38.

In embodiments, the second chemical linker includes a sequence that has at least 90%, 95%, 96%, 97%, 98%, 99% or 100% amino acid sequence identity across the whole sequence or a portion of the sequence (e.g. a 10, 50, 100 continuous amino acid portion) of SEQ ID NO:2, SEQ ID NO:4, SEQ ID NO:6, SEQ ID NO:8, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, or SEQ ID NO:38.

In embodiments, the second chemical linker is the sequence of SEQ ID NO:2. In embodiments, the second chemical linker is the sequence of SEQ ID NO:4. In embodiments, the second chemical linker is the sequence of SEQ ID NO:6. In embodiments, the second chemical linker is the sequence of SEQ ID NO:8. In embodiments, the second chemical linker is the sequence of SEQ ID NO:18. In embodiments, the second chemical linker is the sequence of SEQ ID NO:20. In embodiments, the second chemical linker is the sequence of SEQ ID NO:22. In embodiments, the second chemical linker is the sequence of SEQ ID NO:24. In embodiments, the second chemical linker is the sequence of SEQ ID NO:26. In embodiments, the second chemical linker is the sequence of SEQ ID NO:28. In embodiments, the second chemical linker is the sequence of SEQ ID NO:30. In embodiments, the second chemical linker is the sequence of SEQ ID NO:32. In embodiments, the second chemical linker is the sequence of SEQ ID NO:34. In embodiments, the second chemical linker is the sequence of SEQ ID NO:36. In embodiments, the second chemical linker is the sequence of SEQ ID NO:38.

The ability of an antibody to bind a specific epitope (e.g., HER2) or a ligand binding domain-ligand binding domain enhancer complex (e.g., IL-15-sushi complex) to bind its ligand or target ligand (e.g., IL-15 receptor or subunit thereof) can be described by the equilibrium dissociation constant (K_{D}). The equilibrium dissociation constant (K_{D}) as defined herein is the ratio of the dissociation rate (K-off) and the association rate (K-on) of an antibody to its epitope. The equilibrium dissociation constant (K_{D}) as defined herein is the ratio of the dissociation rate (K-off) and the association rate (K-on) of an ligand binding domain to its ligand. It is described by the following formula: K_{D}=K-off/K-on.

In embodiments, the ligand binding domain (e.g., IL-15) binds the target ligand (IL-15 receptor or subunit thereof) with a K_{D} from 0.1 nM to 40 nM. In embodiments, the ligand binding domain binds the target ligand with a K_{D} from 2 nM to 40 nM. In embodiments, the ligand binding domain binds the target ligand with a K_{D} from 4 nM to 40 nM. In embodiments, the ligand binding domain binds the target ligand with a K_{D} from 6 nM to 40 nM. In embodiments, the ligand binding domain binds the target ligand with a K_{D} from 8 nM to 40 nM. In embodiments, the ligand binding domain binds the target ligand with a K_{D} from 10 nM to 40 nM. In embodiments, the ligand binding domain binds the target ligand with a K_{D} from 12 nM to 40 nM. In embodiments, the ligand binding domain binds the target ligand with a K_{D} from 14 nM to 40 nM. In embodiments, the ligand binding domain binds the target ligand with a K_{D} from 16 nM to 40 nM. In embodiments, the ligand binding domain binds the target ligand with a K_{D} from 18 nM to 40 nM. In embodiments, the ligand binding domain binds the target ligand with a K_{D} from 20 nM to 40 nM. In embodiments, the ligand binding domain binds the target ligand with a K_{D} from 22 nM to 40 nM. In embodiments, the ligand binding domain binds the target ligand with a K_{D} from 24 nM to 40 nM. In embodiments, the ligand binding domain binds the target ligand with a K_{D} from 26 nM to 40 nM. In embodiments, the ligand binding domain binds the target ligand with a K_{D} from 28 nM to 40 nM. In embodiments, the ligand binding domain binds the target ligand with a K_{D} from 30 nM to 40 nM. In embodiments, the ligand binding domain binds the target ligand with a K_{D} from 32 nM to 40 nM. In embodiments, the ligand binding domain binds the target ligand with a K_{D} from 34 nM to 40 nM. In embodiments, the ligand binding domain binds the target ligand with a K_{D} from 36 nM to 40 nM. In embodiments, the ligand binding domain binds the target ligand with a K_{D} from 38 nM to 40 nM.

In embodiments, the ligand binding domain binds the target ligand with a K_{D} from 0.1 nM to 38 nM. In embodiments, the ligand binding domain binds the target ligand with a K_{D} from 0.1 nM to 36 nM. In embodiments, the ligand binding domain binds the target ligand with a K_{D} from 0.1 nM to 34 nM. In embodiments, the ligand binding domain binds the target ligand with a K_{D} from 0.1 nM to 32 nM. In embodiments, the ligand binding domain binds the target ligand with a K_{D} from 0.1 nM to 30 nM. In embodiments, the ligand binding domain binds the target ligand with a K_{D} from 0.1 nM to 28 nM. In embodiments, the ligand binding domain binds the target ligand with a K_{D} from 0.1 nM to 26 nM. In embodiments, the ligand binding domain binds the target ligand with a K_{D} from 0.1 nM to 24 nM. In embodiments, the ligand binding domain binds the target ligand with a K_{D} from 0.1 nM to 22 nM. In embodiments, the ligand binding domain binds the target ligand with a K_{D} from 0.1 nM to 20 nM. In embodiments, the ligand binding domain binds the target ligand with a K_{D} from 0.1 nM to 18 nM. In embodiments, the ligand binding domain binds the target ligand with a K_{D} from 0.1 nM to 16 nM. In embodiments, the ligand binding domain binds the target ligand with a K_{D} from 0.1 nM to 14 nM. In embodiments, the ligand binding domain binds the target ligand with a K_{D} from 0.1 nM to 12 nM. In embodiments, the ligand binding domain binds the target ligand with a K_{D} from 0.1 nM to 10 nM. In embodiments, the ligand binding domain binds the target ligand with a K_{D} from 0.1 nM to 8 nM. In embodiments, the ligand binding domain binds the target ligand with a K_{D} from 0.1 nM to 6 nM. In embodiments, the ligand binding domain binds the target ligand with a K_{D} from 0.1 nM to 4 nM. In embodiments, the ligand binding domain binds the target ligand with a K_{D} from 0.1 nM to 2 nM. In embodiments, the ligand binding domain binds the target ligand with a K_{D} of 0.1 nM, 2 nM, 4 nM, 6 nM, 8 nM, 10 nM, 12 nM, 14 nM, 16 nM, 18 nM, 20 nM, 22 nM, 24 nM, 26 nM, 28 nM, 30 nM, 32 nM, 34 nM, 36 nM, 38 nM or 40 nM. In embodiments, the ligand binding domain binds the target ligand with a K_{D} of 21.9 nM. In embodiments, the ligand binding domain binds the target ligand with a K_{D} of about 21.9 nM. In embodiments, the IL-15 domain binds the IL-15 receptor or subunit thereof with a K_{D} of 0.1 nM, 2 nM, 4 nM, 6 nM, 8 nM, 10 nM, 12 nM, 14 nM, 16 nM, 18 nM, 20 nM, 22 nM, 24 nM, 26 nM, 28 nM, 30 nM, 32 nM, 34 nM, 36 nM, 38 nM or 40 nM. In embodiments, the IL-15 domain binds the IL-15 receptor or subunit thereof with a K_{D} of 21.9 nM. In embodiments, the IL-15 domain binds the IL-15 receptor or subunit thereof with a K_{D} of about 21.9 nM.

In embodiments, the ligand binding domain binds the target ligand with a K_{D} from 0.1 nM to 20 nM. In embodiments, the ligand binding domain binds the target ligand with a K_{D} from 0.5 nM to 20 nM. In embodiments, the ligand binding domain binds the target ligand with a K_{D} from 1 nM to 20 nM. In embodiments, the ligand binding domain binds the target ligand with a K_{D} from 1.5 nM to 20 nM. In embodiments, the ligand binding domain binds the target ligand with a K_{D} from 2 nM to 20 nM. In embodiments, the ligand binding domain binds the target ligand with a K_{D} from 2.5 nM to 20 nM. In embodiments, the ligand binding domain binds the target ligand with a K_{D} from 3 nM to 20 nM. In embodiments, the ligand binding domain binds the target ligand with a K_{D} from 3.5 nM to 20 nM. In embodiments, the ligand binding domain binds the target ligand with a K_{D} from 4 nM to 20 nM. In embodiments, the ligand binding domain binds the target ligand with a K_{D} from 4.5 nM to 20 nM. In embodiments, the ligand binding domain binds the target ligand with a K_{D} from 5 nM to 20 nM. In embodiments, the ligand binding domain binds the target ligand with a K_{D} from 5.5 nM to 20 nM. In embodiments, the ligand binding domain binds the target ligand with a K_{D} from 6 nM to 20 nM.

In embodiments, the ligand binding domain binds the target ligand with a K_{D} from 6.5 nM to 20 nM. In embodiments, the ligand binding domain binds the target ligand with a K_{D} from 7 nM to 20 nM. In embodiments, the ligand binding domain binds the target ligand with a K_{D} from 7.5 nM to 20 nM. In embodiments, the ligand binding domain binds the target ligand with a K_{D} from 8 nM to 20 nM. In embodiments, the ligand binding domain binds the target ligand with a K_{D} from 8.5 nM to 20 nM. In embodiments, the ligand binding domain binds the target ligand with a K_{D} from 9 nM to 20 nM. In embodiments, the ligand binding domain binds the target ligand with a K_{D} from 9.5 nM to 20 nM. In embodiments, the ligand binding domain binds the target ligand with a K_{D} from 10 nM to 20 nM. In embodiments, the ligand binding domain binds the target ligand with a K_{D} from 10.5 nM to 20 nM. In embodiments, the ligand binding domain binds the target ligand with a K_{D} from 11 nM to 20 nM. In embodiments, the ligand binding domain binds the target ligand with a K_{D} from 11.5 nM to 20 nM. In embodiments, the ligand binding domain binds the target ligand with a K_{D} from 12 nM to 20 nM. In embodiments, the ligand binding domain binds the target ligand with a K_{D} from 12.5 nM to 20 nM. In embodiments, the ligand binding domain binds the target ligand with a K_{D} from 13 nM to 20 nM. In embodiments, the ligand binding domain binds the target ligand with a K_{D} from 13.5 nM to 20 nM.

In embodiments, the ligand binding domain binds the target ligand with a K_{D} from 14 nM to 20 nM. In embodiments, the ligand binding domain binds the target ligand with a K_{D} from 14.5 nM to 20 nM. In embodiments, the ligand binding domain binds the target ligand with a K_{D} from 15 nM to 20 nM. In embodiments, the ligand binding domain binds the target ligand with a K_{D} from 15.5 nM to 20 nM. In embodiments, the ligand binding domain binds the target ligand with a K_{D} from 16 nM to 20 nM. In embodiments, the ligand binding domain binds the target ligand with a K_{D} from 16.5 nM to 20 nM. In embodiments, the ligand binding domain binds the target ligand with a K_{D} from 17 nM to 20 nM. In embodiments, the ligand binding domain binds the target ligand with a K_{D} from 17.5 nM to 20 nM. In embodiments, the ligand binding domain binds the target ligand with a K_{D} from 18 nM to 20 nM. In embodiments, the ligand binding domain binds the target ligand with a K_{D} from 18.5 nM to 20 nM. In embodiments, the ligand binding domain binds the target ligand with a K_{D} from 19 nM to 20 nM. In embodiments, the ligand binding domain binds the target ligand with a K_{D} from 19.5 nM to 20 nM.

In embodiments, the ligand binding domain binds the target ligand with a K_{D} from 0.1 nM to 19.5 nM. In embodiments, the ligand binding domain binds the target ligand with a K_{D} from 0.1 nM to 19 nM. In embodiments, the ligand binding domain binds the target ligand with a K_{D} from 0.1 nM to 18.5 nM. In embodiments, the ligand binding domain binds the target ligand with a K_{D} from 0.1 nM to 18 nM. In embodiments, the ligand binding domain binds the target ligand with a K_{D} from 0.1 nM to 17.5 nM. In embodiments, the ligand binding domain binds the target ligand with a K_{D} from 0.1 nM to 17 nM. In embodiments, the ligand binding domain binds the target ligand with a K_{D} from 0.1 nM to 16.5 nM. In embodiments, the ligand binding domain binds the target ligand with a K_{D} from 0.1 nM to 16 nM. In embodiments, the ligand binding domain binds the target ligand with a K_{D} from 0.1 nM to 15.5 nM. In embodiments, the ligand binding domain binds the target ligand with a K_{D} from 0.1 nM to 15 nM. In embodiments, the ligand binding domain binds the target ligand with a K_{D} from 0.1 nM to 14.5 nM. In embodiments, the ligand binding domain binds the target ligand with a K_{D} from 0.1 nM to 14 nM. In embodiments, the ligand binding domain binds the target ligand with a K_{D} from 0.1 nM to 13.5 nM. In embodiments, the ligand binding domain binds the target ligand with a K_{D} from 0.1 nM to 13 nM. In embodiments, the ligand binding domain binds the target ligand with a K_{D} from 0.1 nM to 12.5 nM. In embodiments, the ligand binding domain binds the target ligand with a K_{D} from 0.1 nM to 12 nM. In embodiments, the ligand binding domain binds the target ligand with a K_{D} from 0.1 nM to 11.5 nM. In embodiments, the ligand binding domain binds the target ligand with a K_{D} from 0.1 nM to 11 nM. In embodiments, the ligand binding domain binds the target ligand with a K_{D} from 0.1 nM to 12.5 nM. In embodiments, the ligand binding domain binds the target ligand with a K_{D} from 0.1 nM to 12 nM.

In embodiments, the ligand binding domain binds the target ligand with a K_{D} from 0.1 nM to 11.5 nM. In embodiments, the ligand binding domain binds the target ligand with a K_{D} from 0.1 nM to 11 nM. In embodiments, the ligand binding domain binds the target ligand with a K_{D} from 0.1 nM to 10.5 nM. In embodiments, the ligand binding domain binds the target ligand with a K_{D} from 0.1 nM to 10 nM. In embodiments, the ligand binding domain binds the target ligand with a K_{D} from 0.1 nM to 9.5 nM. In embodiments, the ligand binding domain binds the target ligand with a K_{D} from 0.1 nM to 9 nM. In embodiments, the ligand binding domain binds the target ligand with a K_{D} from 0.1 nM to 8.5 nM. In embodiments, the ligand binding domain binds the target ligand with a K_{D} from 0.1 nM to 8 nM. In embodiments, the ligand binding domain binds the target ligand with a K_{D} from 0.1 nM to 7.5 nM. In embodiments, the ligand binding domain binds the target ligand with a K_{D} from 0.1 nM to 7 nM. In embodiments, the ligand binding domain binds the target ligand with a K_{D} from 0.1 nM to 6.5 nM. In embodiments, the ligand binding domain binds the target ligand with a K_{D} from 0.1 nM to 6 nM. In embodiments, the ligand binding domain binds the target ligand with a K_{D} from 0.1 nM to 5.5 nM. In embodiments, the ligand binding domain binds the target ligand with a K_{D} from 0.1 nM to 5 nM. In embodiments, the ligand binding domain binds the target ligand with a K_{D} from 0.1 nM to 4.5 nM.

In embodiments, the ligand binding domain binds the target ligand with a K_{D} from 0.1 nM to 4 nM. In embodiments, the ligand binding domain binds the target ligand with a K_{D} from 0.1 nM to 3.5 nM. In embodiments, the ligand binding domain binds the target ligand with a K_{D} from 0.1 nM to 3 nM. In embodiments, the ligand binding domain binds the target ligand with a K_{D} from 0.1 nM to 2.5 nM. In embodiments, the ligand binding domain binds the target ligand with a K_{D} from 0.1 nM to 2 nM. In embodiments, the ligand binding domain binds the target ligand with a K_{D} from 0.1 nM to 1.5 nM. In embodiments, the ligand binding domain binds the target ligand with a K_{D} from 0.1 nM to 1 nM. In embodiments, the ligand binding domain binds the target ligand with a K_{D} from 0.1 nM to 0.5 nM. In embodiments, the ligand binding domain binds the target ligand with a K_{D} of 0.1 nM, 0.5 nM, 1 nM, 1.5 nM, 2 nM, 2.5 nM, 3 nM, 3.5 nM, 4 nM, 4.5 nM, 5 nM, 5.5 nM, 6 nM, 6.5 nM, 7 nM, 7.5 nM, 8 nM, 8.5 nM, 9 nM, 9.5 nM, 10 nM, 10.5 nM, 11 nM, 11.5 nM, 12 nM, 12.5 nM, 13 nM, 13.5 nM, 14 nM, 14.5 nM, 15 nM, 15.5 nM, 16 nM, 16.5 nM, 17 nM, 17.5 nM, 18 nM, 18.5 nM, 19 nM, 19.5 nM, or 20 nM. In embodiments, the ligand binding domain binds the target ligand with a K_{D} of 10.7 nM. In embodiments, the ligand binding domain binds the target ligand with a K_{D} of about 10.7 nM. In embodiments, the IL-15 domain binds the IL-15 receptor or subunit thereof with a K_{D} of 0.1 nM, 0.5 nM, 1 nM, 1.5 nM, 2 nM, 2.5 nM, 3 nM, 3.5 nM, 4 nM, 4.5 nM, 5 nM, 5.5 nM, 6 nM, 6.5 nM, 7 nM, 7.5 nM, 8 nM, 8.5 nM, 9 nM, 9.5 nM, 10 nM, 10.5 nM, 11 nM, 11.5 nM, 12 nM, 12.5 nM, 13 nM, 13.5 nM, 14 nM, 14.5 nM, 15 nM, 15.5 nM, 16 nM, 16.5 nM, 17 nM, 17.5 nM, 18 nM, 18.5 nM, 19 nM, 19.5 nM, or 20 nM. In embodiments, the IL-15 domain binds the IL-15 receptor or subunit thereof with a K_{D} of 10.7 nM. In embodiments, the IL-15 domain binds the IL-15 receptor or subunit thereof with a K_{D} of about 10.7 nM.

In embodiments, the ligand binding domain binds the target ligand with a K_{D} from 10 nM to 30 nM. In embodiments, the ligand binding domain binds the target ligand with a K_{D} from 12 nM to 30 nM. In embodiments, the ligand binding domain binds the target ligand with a K_{D} from 14 nM to 30 nM. In embodiments, the ligand binding domain binds the target ligand with a K_{D} from 16 nM to 30 nM. In embodiments, the ligand binding domain binds the target ligand with a K_{D} from 18 nM to 30 nM. In embodiments, the ligand binding domain binds the target ligand with a K_{D} from 20 nM to 30 nM. In embodiments, the ligand binding domain binds the target ligand with a K_{D} from 22 nM to 30 nM. In embodiments, the ligand binding domain binds the target ligand with a K_{D} from 24 nM to 30 nM. In embodiments, the ligand binding domain binds the target ligand with a K_{D} from 26 nM to 30 nM. In embodiments, the ligand binding domain binds the target ligand with a K_{D} from 28 nM to 30 nM.

In embodiments, the ligand binding domain binds the target ligand with a K_{D} from 10 nM to 28 nM. In embodiments, the ligand binding domain binds the target ligand with a K_{D} from 10 nM to 26 nM. In embodiments, the ligand binding domain binds the target ligand with a K_{D} from 10 nM to 24 nM. In embodiments, the ligand binding domain binds the target ligand with a K_{D} from 10 nM to 22 nM. In embodiments, the ligand binding domain binds the target ligand with a K_{D} from 10 nM to 20 nM. In embodiments, the ligand binding domain binds the target ligand with a K_{D} from 10 nM to 18 nM. In embodiments, the ligand binding domain binds the target ligand with a K_{D} from 10 nM to 16 nM. In embodiments, the ligand binding domain binds the target ligand with a K_{D} from 10 nM to 14 nM. In embodiments, the ligand binding domain binds the target ligand with a K_{D} from 10 nM to 12 nM. In embodiments, the ligand binding domain binds the target ligand with a K_{D} of 10 nM, 12 nM, 14 nM, 16 nM, 18 nM, 20 nM, 22 nM, 24 nM, 26 nM, 28 nM, or 30 nM. In embodiments, the ligand binding domain binds the target ligand with a K_{D} of 20.5 nM. In embodiments, the ligand binding domain binds the target ligand with a K_{D} of about 20.5 nM. In embodiments, the ligand binding domain binds the target ligand with a K_{D} of 22.9 nM. In embodiments, the ligand binding domain binds the target ligand with a K_{D} of about 22.9 nM. In embodiments, the IL-15 domain binds the IL-15 receptor or subunit thereof with a K_{D} of 10 nM, 12 nM, 14 nM, 16 nM, 18 nM, 20 nM, 22 nM, 24 nM, 26 nM, 28 nM, or 30 nM. In embodiments, the IL-15 domain binds the IL-15 receptor or subunit thereof with a K_{D} of 20.5 nM. In embodiments, the IL-15 domain binds the IL-15 receptor or subunit thereof with a K_{D} of about 20.5 nM. In embodiments, the ligand binding domain binds the target ligand with a K_{D} of 22.9 nM. In embodiments, the ligand binding domain binds the target ligand with a K_{D} of about 22.9 nM.

In embodiments, the ligand binding domain binds the target ligand with a K_{D} from 0.01 nM to 30 nM. In embodiments, the ligand binding domain binds the target ligand with a K_{D} from 1 nM to 30 nM. In embodiments, the ligand binding domain binds the target ligand with a K_{D} from 2 nM to 30 nM. In embodiments, the ligand binding domain binds the target ligand with a K_{D} from 3 nM to 30 nM. In embodiments, the ligand binding domain binds the target ligand with a K_{D} from 4 nM to 30 nM. In embodiments, the ligand binding domain binds the target ligand with a K_{D} from 5 nM to 30 nM. In embodiments, the ligand binding domain binds the target ligand with a K_{D} from 6 nM to 30 nM. In embodiments, the ligand binding domain binds the target ligand with a K_{D} from 7 nM to 30 nM. In embodiments, the ligand binding domain binds the target ligand with a K_{D} from 8 nM to 30 nM. In embodiments, the ligand binding domain binds the target ligand with a K_{D} from 9 nM to 30 nM. In embodiments, the ligand binding domain binds the target ligand with a K_{D} from 10 nM to 30 nM. In embodiments, the ligand binding domain binds the target ligand with a K_{D} from 11 nM to 30 nM. In embodiments, the ligand binding domain binds the target ligand with a K_{D} from 12 nM to 30 nM.

In embodiments, the ligand binding domain binds the target ligand with a K_{D} from 13 nM to 30 nM. In embodiments, the ligand binding domain binds the target ligand with a K_{D} from 14 nM to 30 nM. In embodiments, the ligand binding domain binds the target ligand with a K_{D} from 15 nM to 30 nM. In embodiments, the ligand binding domain binds the target ligand with a K_{D} from 16 nM to 30 nM. In embodiments, the ligand binding domain binds the target ligand with a K_{D} from 17 nM to 30 nM. In embodiments, the ligand binding domain binds the target ligand with a K_{D} from 18 nM to 30 nM. In embodiments, the ligand binding domain binds the target ligand with a K_{D} from 19 nM to 30 nM. In embodiments, the ligand binding domain binds a target ligand with a K_{D} from 20 nM to 30 nM. In embodiments, the ligand binding domain binds a target ligand with a K_{D} from 21 nM to 30 nM. In embodiments, the ligand binding domain binds a target ligand with a K_{D} from 22 nM to 30 nM. In embodiments, the ligand binding domain binds a target ligand with a K_{D} from 23 nM to 30 nM. In embodiments, the ligand binding domain binds a target ligand with a K_{D} from 24 nM to 30 nM. In embodiments, the ligand binding domain binds a target ligand with a K_{D} from 25 nM to 30 nM. In embodiments, the ligand binding domain binds a target ligand with a K_{D} from 26 nM to 30 nM. In embodiments, the ligand binding domain binds the target ligand with a K_{D} from 27 nM to 30 nM. In embodiments, the ligand binding domain binds the target ligand with a K_{D} from 28 nM to 30 nM. In embodiments, the ligand binding domain binds the target ligand with a K_{D} from 29 nM to 30 nM.

In embodiments, the ligand binding domain binds the target ligand with a K_{D} from 0.01 nM to 29 nM. In embodiments, the ligand binding domain binds the target ligand with a K_{D} from 0.01 nM to 28 nM. In embodiments, the ligand binding domain binds the target ligand with a K_{D} from 0.01 nM to 27 nM. In embodiments, the ligand binding domain binds the target ligand with a K_{D} from 0.01 nM to 26 nM. In embodiments, the ligand binding domain binds the target ligand with a K_{D} from 0.01 nM to 25 nM. In embodiments, the ligand binding domain binds the target ligand with a K_{D} from 0.01 nM to 24 nM. In embodiments, the ligand binding domain binds the target ligand with a K_{D} from 0.01 nM to 23 nM. In embodiments, the ligand binding domain binds the target ligand with a K_{D} from 0.01 nM to 22 nM. In embodiments, the ligand binding domain binds the target ligand with a K_{D} from 0.01 nM to 21 nM. In embodiments, the ligand binding domain binds the target ligand with a K_{D} from 0.01 nM to 20 nM. In embodiments, the ligand binding domain binds the target ligand with a K_{D} from 0.01 nM to 19 nM. In embodiments, the ligand binding domain binds the target ligand with a K_{D} from 0.01 nM to 18 nM. In embodiments, the ligand binding domain binds the target ligand with a K_{D} from 0.01 nM to 17 nM.

In embodiments, the ligand binding domain binds the target ligand with a K_{D} from 0.01 nM to 16 nM. In embodiments, the ligand binding domain binds the target ligand with a K_{D} from 0.01 nM to 15 nM. In embodiments, the ligand binding domain binds the target ligand with a K_{D} from 0.01 nM to 14 nM. In embodiments, the ligand binding domain binds the target ligand with a K_{D} from 0.01 nM to 13 nM. In embodiments, the ligand binding domain binds the target ligand with a K_{D} from 0.01 nM to 12 nM. In embodiments, the ligand binding domain binds the target ligand with a K_{D} from 0.01 nM to 11 nM. In embodiments, the ligand binding domain binds the target ligand with a K_{D} from 0.01 nM to 10 nM. In embodiments, the ligand binding domain binds the target ligand with a K_{D} from 0.01 nM to 9 nM. In embodiments, the ligand binding domain binds the target ligand with a K_{D} from 0.01 nM to 8 nM. In embodiments, the ligand binding domain binds the target ligand with a K_{D} from 0.01 nM to 7 nM. In embodiments, the ligand binding domain binds the target ligand with a K_{D} from 0.01 nM to 6 nM. In embodiments, the ligand binding domain binds the target ligand with a K_{D} from 0.01 nM to 5 nM. In embodiments, the ligand binding domain binds the target ligand with a K_{D} from 0.01 nM to 4 nM. In embodiments, the ligand binding domain binds the target ligand with a K_{D} from 0.01 nM to 3 nM.

In embodiments, the ligand binding domain binds the target ligand with a K_{D} from 0.01 nM to 2 nM. In embodiments, the ligand binding domain binds the target ligand with a K_{D} from 0.01 nM to 1 nM. In embodiments, the ligand binding domain binds the target ligand with a K_{D} of 0.01 nM, 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM or 30 nM. In embodiments, the ligand binding domain binds the target ligand with a K_{D} of 10.1 nM. In embodiments, the ligand binding domain binds the target ligand with a K_{D} of about 10.1 nM. In embodiments, the IL-15 domain binds the IL-15 receptor or subunit thereof with a K_{D} of 0.01 nM, 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM or 30 nM. In embodiments, the IL-15 domain binds the IL-15 receptor or subunit thereof with a K_{D} of 10.1 nM. In embodiments, the IL-15 domain binds the IL-15 receptor or subunit thereof with a K_{D} of about 10.1 nM.

In embodiments, the ligand binding domain binds the target ligand with a K_{D} from 10 nM to 50 nM. In embodiments, the ligand binding domain binds the target ligand with a K_{D} from 12 nM to 50 nM. In embodiments, the ligand binding domain binds the target ligand with a K_{D} from 14 nM to 50 nM. In embodiments, the ligand binding domain binds the target ligand with a K_{D} from 16 nM to 50 nM. In embodiments, the ligand binding domain binds the target ligand with a K_{D} from 18 nM to 50 nM. In embodiments, the ligand binding domain binds the target ligand with a K_{D} from 20 nM to 50 nM. In embodiments, the ligand binding domain binds the target ligand with a K_{D} from 22 nM to 50 nM. In embodiments, the ligand binding domain binds the target ligand with a K_{D} from 24 nM to 50 nM. In embodiments, the ligand binding domain binds the target ligand with a K_{D} from 26 nM to 50 nM. In embodiments, the ligand binding domain binds the target ligand with a K_{D} from 28 nM to 50 nM. In embodiments, the ligand binding domain binds the target ligand with a K_{D} from 30 nM to 50 nM. In embodiments, the ligand binding domain binds the target ligand with a K_{D} from 32 nM to 50 nM. In embodiments, the ligand binding domain binds the target ligand with a K_{D} from 34 nM to 50 nM. In embodiments, the ligand binding domain binds the target ligand with a K_{D} from 36 nM to 50 nM. In embodiments, the ligand binding domain binds the target ligand with a K_{D} from 38 nM to 50 nM. In embodiments, the ligand binding domain binds the target ligand with a K_{D} from 40 nM to 50 nM. In embodiments, the ligand binding domain binds the target ligand with a K_{D} from 42 nM to 50 nM. In embodiments, the ligand binding domain binds the target ligand with a K_{D} from 44 nM to 50 nM. In embodiments, the ligand binding domain binds the target ligand with a K_{D} from 46 nM to 50 nM. In embodiments, the ligand binding domain binds the target ligand with a K_{D} from 48 nM to 50 nM.

In embodiments, the ligand binding domain binds the target ligand with a K_{D} from 10 nM to 48 nM. In embodiments, the ligand binding domain binds the target ligand with a K_{D} from 10 nM to 46 nM. In embodiments, the ligand binding domain binds the target ligand with a K_{D} from 10 nM to 44 nM. In embodiments, the ligand binding domain binds the target ligand with a K_{D} from 10 nM to 42 nM. In embodiments, the ligand binding domain binds the target ligand with a K_{D} from 10 nM to 40 nM. In embodiments, the ligand binding domain binds the target ligand with a K_{D} from 10 nM to 38 nM. In embodiments, the ligand binding domain binds the target ligand with a K_{D} from 10 nM to 36 nM. In embodiments, the ligand binding domain binds the target ligand with a K_{D} from 10 nM to 34 nM. In embodiments, the ligand binding domain binds the target ligand with a K_{D} from 10 nM to 32 nM. In embodiments, the ligand binding domain binds the target ligand with a K_{D} from 10 nM to 30 nM. In embodiments, the ligand binding domain binds the target ligand with a K_{D} from 10 nM to 28 nM. In embodiments, the ligand binding domain binds the target ligand with a K_{D} from 10 nM to 26 nM. In embodiments, the ligand binding domain binds the target ligand with a K_{D} from 10 nM to 24 nM. In embodiments, the ligand binding domain binds the target ligand with a K_{D} from 10 nM to 22 nM. In embodiments, the ligand binding domain binds the target ligand with a K_{D} from 10 nM to 20 nM. In embodiments, the ligand binding domain binds the target ligand with a K_{D} from 10 nM to 18 nM. In embodiments, the ligand binding domain binds the target ligand with a K_{D} from 10 nM to 16 nM.

In embodiments, the ligand binding domain binds the target ligand with a K_{D} from 10 nM to 14 nM. In embodiments, the ligand binding domain binds the target ligand with a K_{D} from 10 nM to 12 nM. In embodiments, the ligand binding domain binds the target ligand with a K_{D} of 10 nM, 12 nM, 14 nM, 16 nM, 18 nM, 20 nM, 22 nM, 24 nM, 26 nM, 28 nM, 30 nM, 32 nM, 34 nM, 36 nM, 38 nM, 40 nM, 42 nM, 44 nM, 46 nM, 48 nM, or 50 nM. In embodiments, the ligand binding domain binds a target ligand with a K_{D} of 33.4 nM. In embodiments, the ligand binding domain binds a target ligand with a K_{D} of about 33.4 nM. In embodiments, the IL-15 domain binds the IL-15 receptor or subunit thereof with a K_{D} of 10 nM, 12 nM, 14 nM, 16 nM, 18 nM, 20 nM, 22 nM, 24 nM, 26 nM, 28 nM, 30 nM, 32 nM, 34 nM, 36 nM, 38 nM, 40 nM, 42 nM, 44 nM, 46 nM, 48 nM, or 50 nM. In embodiments, the IL-15 domain binds the IL-15 receptor or subunit thereof binds a target ligand with a K_{D} of 33.4 nM. In embodiments, the IL-15 domain binds the IL-15 receptor or subunit thereof binds a target ligand with a K_{D} of about 33.4 nM.

In one embodiment, the first protein dimerizing domain is an antibody light chain with the sequence of SEQ ID NO:10, the second protein dimerizing domain is an antibody heavy chain with the sequence of SEQ ID NO:39, the second ligand binding domain is an IL-2 domain with the sequence of SEQ ID NO:40, the first chemical linker has the sequence of SEQ ID NO:1, the second ligand binding domain enhancer is a IL-2 domain enhancer with the sequence of SEQ ID NO:41 and the second chemical linker has the sequence of SEQ ID NO:2, wherein the first chemical linker is attached to the N-terminus of the first protein dimerizing domain and the second chemical linker is attached to the C-terminus of the first protein dimerizing domain.

In one embodiment, the first protein dimerizing domain is an antibody heavy chain with the sequence of SEQ ID NO:39, the second protein dimerizing domain is an antibody light chain with the sequence of SEQ ID NO:10, the second ligand binding domain is an IL-2 domain with the sequence of SEQ ID NO:40, the first chemical linker has the sequence of SEQ ID NO:3, the second ligand binding domain enhancer is a IL-2 domain enhancer with the sequence of SEQ ID NO:41 and the second chemical linker has the sequence of SEQ ID NO:4, wherein the first chemical linker is attached to the N-terminus of the first protein dimerizing domain and the second chemical linker is attached to the C-terminus of the first protein dimerizing domain.

In one embodiment, the first protein dimerizing domain is an antibody light chain with the sequence of SEQ ID NO:10, the second protein dimerizing domain is an antibody heavy chain with the sequence of SEQ ID NO:39, the second ligand binding domain is an IL-2 domain with the sequence of SEQ ID NO:40, the first chemical linker has the sequence of SEQ ID NO:5, the second ligand binding domain enhancer is a IL-2 domain enhancer with the sequence of SEQ ID NO:41 and the second chemical linker has the sequence of SEQ ID NO:6, wherein the first chemical linker is attached to the C-terminus of the first protein dimerizing domain and the second chemical linker is attached to the N-terminus of the first protein dimerizing domain.

In one embodiment, the first protein dimerizing domain is an antibody heavy chain with the sequence of SEQ ID NO:39, the second protein dimerizing domain is an antibody light chain with the sequence of SEQ ID NO:10, the second ligand binding domain is an IL-2 domain with the sequence of SEQ ID NO:40, the first chemical linker has the sequence of SEQ ID NO:7, the second ligand binding domain enhancer is a IL-2 domain enhancer with the sequence of SEQ ID NO:41 and the second chemical linker has the sequence of SEQ ID NO:8, wherein the first chemical linker is attached to the C-terminus of the first protein dimerizing domain and the second chemical linker is attached to the N-terminus of the first protein dimerizing domain.

In one embodiment, the first protein dimerizing domain is an antibody heavy chain with the sequence of SEQ ID NO:39, the second protein dimerizing domain is an antibody light chain with the sequence of SEQ ID NO:10, the second ligand binding domain is an IL-15 domain with the sequence of SEQ ID NO:42, the first chemical linker has the sequence of SEQ ID NO:17, the second ligand binding domain enhancer is a IL-15 domain enhancer with the sequence of SEQ ID NO:43 and the second chemical linker has the sequence of SEQ ID NO:18, wherein the first chemical linker is attached to the N-terminus of the first protein dimerizing domain and the second chemical linker is attached to the C-terminus of the first protein dimerizing domain.

In one embodiment, the first protein dimerizing domain is an antibody heavy chain with the sequence of SEQ ID NO:39, the second protein dimerizing domain is an antibody light chain with the sequence of SEQ ID NO:10, the second ligand binding domain is an IL-15 domain with the sequence of SEQ ID NO:42, the first chemical linker has the sequence of SEQ ID NO:19, the second ligand binding domain enhancer is a IL-15 domain enhancer with the sequence of SEQ ID NO:43 and the second chemical linker has the sequence of SEQ ID NO:20, wherein the first chemical linker is attached to the N-terminus of the first protein dimerizing domain and the second chemical linker is attached to the C-terminus of the first protein dimerizing domain.

In one embodiment, the first protein dimerizing domain is an antibody heavy chain with the sequence of SEQ ID NO:39, the second protein dimerizing domain is an antibody light chain with the sequence of SEQ ID NO:10, the second ligand binding domain is an IL-15 domain with the sequence of SEQ ID NO:45, the first chemical linker has the sequence of SEQ ID NO:21, the second ligand binding domain enhancer is a IL-15 domain enhancer with the sequence of SEQ ID NO:44 and the second chemical linker has the sequence of SEQ ID NO:22, wherein the first chemical linker is attached to the N-terminus of the first protein dimerizing domain and the second chemical linker is attached to the C-terminus of the first protein dimerizing domain.

In one embodiment, the first protein dimerizing domain is an antibody heavy chain with the sequence of SEQ ID NO:39, the second protein dimerizing domain is an antibody light chain with the sequence of SEQ ID NO:10, the second ligand binding domain is an IL-15 domain with the sequence of SEQ ID NO:45, the first chemical linker has the sequence of SEQ ID NO:23, the second ligand binding domain enhancer is a IL-15 domain enhancer with the sequence of SEQ ID NO:44 and the second chemical linker has the sequence of SEQ ID NO:24, wherein the first chemical linker is attached to the N-terminus of the first protein dimerizing domain and the second chemical linker is attached to the C-terminus of the first protein dimerizing domain.

In one embodiment, the first protein dimerizing domain is an antibody heavy chain with the sequence of SEQ ID NO:39, the second protein dimerizing domain is an antibody light chain with the sequence of SEQ ID NO:10, the second ligand binding domain is an IL-15 domain with the sequence of SEQ ID NO:42, the first chemical linker has the sequence of SEQ ID NO:25, the second ligand binding domain enhancer is a IL-15 domain enhancer with the sequence of SEQ ID NO:43 and the second chemical linker has the sequence of SEQ ID NO:26, wherein the first chemical linker is attached to the N-terminus of the first protein dimerizing domain and the second chemical linker is attached to the C-terminus of the first protein dimerizing domain.

In one embodiment, the first protein dimerizing domain is an antibody heavy chain with the sequence of SEQ ID NO:39, the second protein dimerizing domain is an antibody light chain with the sequence of SEQ ID NO:10, the second ligand binding domain is an IL-15 domain with the sequence of SEQ ID NO:42, the first chemical linker has the sequence of SEQ ID NO:27, the second ligand binding domain enhancer is a IL-15 domain enhancer with the sequence of SEQ ID NO:43 and the second chemical linker has the sequence of SEQ ID NO:28, wherein the first chemical linker is attached to the N-terminus of the first protein dimerizing domain and the second chemical linker is attached to the C-terminus of the first protein dimerizing domain.

In one embodiment, the first protein dimerizing domain is an antibody heavy chain with the sequence of SEQ ID NO:39, the second protein dimerizing domain is an antibody light chain with the sequence of SEQ ID NO:10, the second ligand binding domain is an IL-15 domain with the sequence of SEQ ID NO:42, the first chemical linker has the sequence of SEQ ID NO:29, the second ligand binding domain enhancer is a IL-15 domain enhancer with the sequence of SEQ ID NO:43 and the second chemical linker has the sequence of SEQ ID NO:30, wherein the first chemical linker is attached to the N-terminus of the first protein dimerizing domain and the second chemical linker is attached to the C-terminus of the first protein dimerizing domain.

In one embodiment, the first protein dimerizing domain is an antibody heavy chain with the sequence of SEQ ID NO:39, the second protein dimerizing domain is an antibody light chain with the sequence of SEQ ID NO:10, the second ligand binding domain is an IL-15 domain with the sequence of SEQ ID NO:42, the first chemical linker has the sequence of SEQ ID NO:31, the second ligand binding domain enhancer is a IL-15 domain enhancer with the sequence of SEQ ID NO:43 and the second chemical linker has the sequence of SEQ ID NO:32, wherein the first chemical linker is attached to the N-terminus of the first protein dimerizing domain and the second chemical linker is attached to the C-terminus of the first protein dimerizing domain.

In one embodiment, the first protein dimerizing domain is an antibody heavy chain with the sequence of SEQ ID NO:39, the second protein dimerizing domain is an antibody light chain with the sequence of SEQ ID NO:10, the second ligand binding domain is an IL-15 domain with the sequence of SEQ ID NO:42, the first chemical linker has the sequence of SEQ ID NO:33, the second ligand binding domain enhancer is a IL-15 domain enhancer with the sequence of SEQ ID NO:43 and the second chemical linker has the sequence of SEQ ID NO:34, wherein the first chemical linker is attached to the N-terminus of the first protein dimerizing domain and the second chemical linker is attached to the C-terminus of the first protein dimerizing domain.

In one embodiment, the first protein dimerizing domain is an antibody light chain with the sequence of SEQ ID NO:10, the second protein dimerizing domain is an antibody heavy chain with the sequence of SEQ ID NO:39, the second ligand binding domain is an IL-15 domain with the sequence of SEQ ID NO:42, the first chemical linker has the sequence of SEQ ID NO:35, the second ligand binding domain enhancer is a IL-15 domain enhancer with the sequence of SEQ ID NO:43 and the second chemical linker has the sequence of SEQ ID NO:36, wherein the first chemical linker is attached to the C-terminus of the first protein dimerizing domain and the second chemical linker is attached to the N-terminus of the first protein dimerizing domain.

In one embodiment, the first protein dimerizing domain is an antibody light chain with the sequence of SEQ ID NO:10, the second protein dimerizing domain is an antibody heavy chain with the sequence of SEQ ID NO:39, the second ligand binding domain is an IL-15 domain with the sequence of SEQ ID NO:42, the first chemical linker has the sequence of SEQ ID NO:37, the second ligand binding domain enhancer is a IL-15 domain enhancer with the sequence of SEQ ID NO:43 and the second chemical linker has the sequence of SEQ ID NO:38, wherein the first chemical linker is attached to the C-terminus of the first protein dimerizing domain and the second chemical linker is attached to the N-terminus of the first protein dimerizing domain.

In embodiments, the multivalent complex provided herein including embodiments thereof binds a target ligand expressed on a cell. In embodiments, the target ligand is an interleukin receptor. In embodiments, the interleukin receptor is an IL-15 receptor. In embodiments, the interleukin receptor is an IL-2 receptor. In embodiments, the cell is an immune cell. In embodiments, the immune cell is a T cell. In embodiments, the immune cell is an Nk cell. In embodiments, the cell is a cancer cell. In embodiments, the cell is a breast cancer cell.

### Nucleic Acid Compositions

The compositions provided herein include nucleic acid molecules encoding the complex or portions thereof provided herein including embodiments thereof. The complexes encoded by the isolated nucleic acid provided herein are described in detail throughout this application (including the description above and in the examples section). Thus, in an aspect, an isolated nucleic acid encoding a multivalent complex or portions thereof as provided herein including embodiments thereof is provided.

### Covalent Complexes

Provided herein are, inter alia, covalent complexes wherein the ligand binding domain and the ligand binding domain enhancer are covalently bound together through a disulfide linkage. The covalent complexes provided herein may, inter alia, be used for therapeutic purposes such as cancer treatment. Thus, in an aspect is provided a covalent complex including a ligand binding domain covalently bound to a ligand binding domain enhancer through one or more disulfide linkages. For the purpose of this invention, the same definitions apply to the ligand binding domain and the ligand binding domain enhancer of the covalent complexes as for the ligand binding domain and the ligand binding domain enhancer of the multivalent complexes provided herein.

In embodiments, the covalent complex consists essentially of the ligand binding domain covalently bound to the ligand binding domain enhancer through one or more disulfide linkages. Where the covalent complex consists essentially of the ligand binding domain covalently bound to the ligand binding domain enhancer, the complex does not include any additional essential components other than the ligand binding domain covalently bound to the ligand binding domain enhancer.

The covalent complexes provided herein may include a ligand binding domain (e.g., IL-15) covalently attached to a ligand binding domain enhancer. The ligand binding domain enhancer increases the stability of the ligand binding domain (e.g., IL-15, IL-2) and its affinity to its innate receptor. The ligand binding domain enhancer may also reduce the entropy of the ligand binding domain (e.g., IL-15, IL-2) relative to the absence of the ligand binding domain (e.g., IL-15, IL-2). The ligand binding domain enhancer may be an IL-15RA receptor domain or an IL-2RA receptor domain. In embodiments, the ligand binding domain enhancer is a sushi domain (an extracellular domain of IL-15RA or IL-2RA).

As defined herein, the term “enhancing”, “enhancer”, and the like in reference to a ligand binding domain enhancer provided herein means positively affecting the biological function (e.g., by increasing binding, or targeted delivery) of the ligand binding domain, which the enhancer binds to. In some embodiments, enhancing refers to the ability to increase the binding affinity or the structural stability of the ligand binding domain relative to the absence of the enhancer. In some embodiments, enhancing refers to increasing the target specificity and the targeted delivery of the ligand binding domain relative to the absence of the enhancer. In some embodiments, enhancing refers to the decrease of unspecific binding of the ligand binding domain relative to the absence of the enhancer. Thus, enhancing includes, at least in part, partially or totally increasing activity, target specificity, or binding affinity of a ligand binding domain relative to the absence of the ligand binding domain enhancer. In embodiments, the covalent complexes provided herein are administered to a subject in need for therapeutic treatment (e.g., cancer treatment) and the ligand binding domain enhancer increases the targeted delivery of the ligand binding domain relative to the absence of ligand binding domain enhancer.

In embodiments, the ligand binding domain is an interleukin domain. In embodiments, the ligand binding domain is an interleukin domain. In embodiments, the ligand binding domain is an IL-2 domain, an IL-4 domain, an IL-7 domain, an IL-9 domain, an IL-15 domain, an IL-21 domain or a thymic stromal lymphopoietin (TSLP) domain. In embodiments, the ligand binding domain is an IL-2 domain. In embodiments, the ligand binding domain is an IL-4 domain. In embodiments, the ligand binding domain is an IL-7 domain. In embodiments, the ligand binding domain is an IL-9 domain. In embodiments, the ligand binding domain is an IL-15 domain. In embodiments, the ligand binding domain is an IL-21 domain. In embodiments, the ligand binding domain is a thymic stromal lymphopoietin (TSLP) domain.

In embodiments, the IL-15 domain includes the sequence of SEQ ID NO:42. In embodiments, the IL-15 domain is the sequence of SEQ ID NO:42. In embodiments, the IL15 domain includes a sequence that has at least 90%, 95%, 96%, 97%, 98%, 99% or 100% amino acid sequence identity across the whole sequence or a portion of the sequence (e.g. a 10, 50, 100 continuous amino acid portion) of SEQ ID NO:42. In embodiments, the IL-15 domain includes the sequence of SEQ ID NO:45. In embodiments, the IL-15 domain is the sequence of SEQ ID NO:45. In embodiments, the IL15 domain includes a sequence that has at least 90%, 95%, 96%, 97%, 98%, 99% or 100% amino acid sequence identity across the whole sequence or a portion of the sequence (e.g. a 10, 50, 100 continuous amino acid portion) of SEQ ID NO:45.

In embodiments, the IL-2 domain includes the sequence of SEQ ID NO:40. In embodiments, the IL-2 domain is the sequence of SEQ ID NO:40. In embodiments, the IL-2 domain includes a sequence that has at least 90%, 95%, 96%, 97%, 98%, 99% or 100% amino acid sequence identity across the whole sequence or a portion of the sequence (e.g. a 10, 50, 100 continuous amino acid portion) of SEQ ID NO:40.

In embodiments, the ligand binding domain enhancer is a chemokine domain enhancer. In embodiments, the ligand binding domain enhancer is an interleukin domain enhancer. In embodiments, the ligand binding domain enhancer includes a sushi domain. In embodiments, the ligand binding domain enhancer is an IL-2 domain enhancer, an IL-4 domain enhancer, an IL-7 domain enhancer, an IL-9 domain enhancer, an IL-15 domain enhancer, an IL-21 domain enhancer or a thymic stromal lymphopoietin (TSLP) domain enhancer. In embodiments, the ligand binding domain enhancer is an IL-2 domain enhancer. In embodiments, the ligand binding domain enhancer is an IL-4 domain enhancer. In embodiments, the ligand binding domain enhancer is an IL-7 domain enhancer. In embodiments, the ligand binding domain enhancer is an IL-9 domain enhancer. In embodiments, the ligand binding domain enhancer is an IL-15 domain enhancer. In embodiments, the ligand binding domain enhancer is an IL-21 domain enhancer. In embodiments, the ligand binding domain enhancer is on thymic stromal lymphopoietin (TSLP) domain enhancer.

In embodiments, the IL-15 domain enhancer includes the sequence of SEQ ID NO:43. In embodiments, the IL-15 domain is the sequence of SEQ ID NO:43. In embodiments, the IL15 domain includes a sequence that has at least 90%, 95%, 96%, 97%, 98%, 99% or 100% amino acid sequence identity across the whole sequence or a portion of the sequence (e.g. a 10, 50, 100 continuous amino acid portion) of SEQ ID NO:43. In embodiments, the IL-15 domain enhancer includes the sequence of SEQ ID NO:44. In embodiments, the IL-15 domain is the sequence of SEQ ID NO:44. In embodiments, the IL15 domain includes a sequence that has at least 90%, 95%, 96%, 97%, 98%, 99% or 100% amino acid sequence identity across the whole sequence or a portion of the sequence (e.g. a 10, 50, 100 continuous amino acid portion) of SEQ ID NO:44.

In embodiments, the IL-2 domain enhancer includes the sequence of SEQ ID NO:41. In embodiments, the IL-2 domain enhancer is the sequence of SEQ ID NO:41. In embodiments, the IL-2 domain enhancer includes a sequence that has at least 90%, 95%, 96%, 97%, 98%, 99% or 100% amino acid sequence identity across the whole sequence or a portion of the sequence (e.g. a 10, 50, 100 continuous amino acid portion) of SEQ ID NO:41.

In embodiments, the ligand binding domain includes a cysteine at a position corresponding to position 45, 87 or 90 of the ligand binding domain. In embodiments, the ligand binding domain includes a cysteine at a position corresponding to position 45, 87 or 90 of the sequence of SEQ ID NO:42. In embodiments, the ligand binding domain includes a cysteine at a position corresponding to position 45 of the ligand binding domain. In embodiments, the ligand binding domain includes a cysteine at a position corresponding to position 87 of the ligand binding domain. In embodiments, the ligand binding domain includes a cysteine at a position corresponding to position 90 of the ligand binding domain. In embodiments, the ligand binding domain includes a cysteine at a position corresponding to position 45 of the sequence of SEQ ID NO:42. In embodiments, the ligand binding domain includes a cysteine at a position corresponding to position 87 of the sequence of SEQ ID NO:42. In embodiments, the ligand binding domain includes a cysteine at a position corresponding to position 90 of the sequence of SEQ ID NO:42.

In embodiments, the ligand binding domain is an interleukin domain. In embodiments, the ligand binding domain is an IL-15 domain. In embodiments, the IL-15 domain includes a cysteine at a position corresponding to position 45, 87 or 90 of the IL-15 domain. In embodiments, the IL-15 domain includes a cysteine at a position corresponding to position 45 of the IL-15 domain. In embodiments, the IL-15 domain includes a cysteine at a position corresponding to position 87 of the IL-15 domain. In embodiments, the IL-15 domain includes a cysteine at a position corresponding to position 90 of the IL-15 domain.

In embodiments, the IL-15 domain includes a cysteine at a position corresponding to position 45 of the sequence of SEQ ID NO:42. In embodiments, the IL-15 domain includes a cysteine at a position corresponding to position 87 of the sequence of SEQ ID NO:42. In embodiments, the IL-15 domain includes a cysteine at a position corresponding to position 90 of the sequence of SEQ ID NO:42.

In embodiments, the ligand binding domain enhancer includes a cysteine at a position corresponding to position 37, 38, 68 or 67 of the ligand binding domain enhancer. In embodiments, the ligand binding domain enhancer includes a cysteine at a position corresponding to position 37 of the ligand binding domain enhancer. In embodiments, the ligand binding domain enhancer includes a cysteine at a position corresponding to position 38 of the ligand binding domain enhancer. In embodiments, the ligand binding domain enhancer includes a cysteine at a position corresponding to position 68 of the ligand binding domain enhancer. In embodiments, the ligand binding domain enhancer includes a cysteine at a position corresponding to position 67 of the ligand binding domain enhancer.

In embodiments, the ligand binding domain enhancer includes a cysteine at a position corresponding to position 37 of the sequence of SEQ ID NO:43. In embodiments, the ligand binding domain enhancer includes a cysteine at a position corresponding to position 38 of the sequence of SEQ ID NO:43. In embodiments, the ligand binding domain enhancer includes a cysteine at a position corresponding to position 68 of the sequence of SEQ ID NO:43. In embodiments, the ligand binding domain enhancer includes a cysteine at a position corresponding to position 67 of the sequence of SEQ ID NO:43.

In embodiments, the ligand binding domain enhancer is an IL-15 domain enhancer. In embodiments, the IL-15 domain enhancer includes a cysteine at a position corresponding to position 37, 38, 68 or 67 of the IL-15 domain enhancer. In embodiments, the IL-15 domain enhancer includes a cysteine at a position corresponding to position 37 of the IL-15 domain enhancer. In embodiments, the IL-15 domain enhancer includes a cysteine at a position corresponding to position 38 of the IL-15 domain enhancer. In embodiments, the IL-15 domain enhancer includes a cysteine at a position corresponding to position 68 of the IL-15 domain enhancer. In embodiments, the IL-15 domain enhancer includes a cysteine at a position corresponding to position 67 of the IL-15 domain enhancer.

In embodiments, the IL-15 domain enhancer includes a cysteine at a position corresponding to position 37 of the sequence of SEQ ID NO:43. In embodiments, the IL-15 domain enhancer includes a cysteine at a position corresponding to position 38 of the sequence of SEQ ID NO:43. In embodiments, the IL-15 domain enhancer includes a cysteine at a position corresponding to position 68 of the sequence of SEQ ID NO:43. In embodiments, the IL-15 domain enhancer includes a cysteine at a position corresponding to position 67 of the sequence of SEQ ID NO:43.

In embodiments, the sushi domain includes a cysteine at a position corresponding to position 37 of the sequence of SEQ ID NO:43. In embodiments, the sushi domain includes a cysteine at a position corresponding to position 38 of the sequence of SEQ ID NO:43. In embodiments, the sushi domain includes a cysteine at a position corresponding to position 68 of the sequence of SEQ ID NO:43. In embodiments, the sushi domain includes a cysteine at a position corresponding to position 67 of the sequence of SEQ ID NO:43.

The one or more disulfide linkages may be formed between any cysteine residues included in the ligand binding domain or the ligand binding domain enhancer. In one embodiment, the disulfide linkage is between a first cysteine at a position corresponding to position 45 of the ligand binding domain and a second cysteine at a position corresponding to position 37 of the ligand binding domain enhancer. In a further embodiment, the ligand binding domain is an IL-15 domain and ligand binding domain enhancer is an IL-15 domain enhancer.

In one embodiment, the disulfide linkage is between a first cysteine at a position corresponding to position 45 of the ligand binding domain and a second cysteine at a position corresponding to position 38 of the ligand binding domain enhancer. In a further embodiment, the ligand binding domain is an IL-15 domain and ligand binding domain enhancer is an IL-15 domain enhancer.

In one embodiment, the disulfide linkage is between a first cysteine at a position corresponding to position 90 of the ligand binding domain and a second cysteine at a position corresponding to position 68 of the ligand binding domain enhancer. In a further embodiment, the ligand binding domain is an IL-15 domain and ligand binding domain enhancer is an IL-15 domain enhancer.

In one embodiment, the disulfide linkage is between a first cysteine at a position corresponding to position 90 of the ligand binding domain and a second cysteine at a position corresponding to position 67 of the ligand binding domain enhancer. In a further embodiment, the ligand binding domain is an IL-15 domain and ligand binding domain enhancer is an IL-15 domain enhancer. In further embodiments, the ligand binding domain includes the sequence of SEQ ID NO:45 and ligand binding domain enhancer includes the sequence of SEQ ID NO:44. In further embodiments, the ligand binding domain has the sequence of SEQ ID NO:45 and ligand binding domain enhancer has the sequence of SEQ ID NO:44. In embodiments, the covalent complexes have an increased melting temperature relative to a standard control. In embodiments, the covalent complexes have an increased melting temperature relative to the absence of the disulfide linkage.

In one embodiment, the disulfide linkage is between a first cysteine at a position corresponding to position 87 of the ligand binding domain and a second cysteine at a position corresponding to position 67 of the ligand binding domain enhancer. In a further embodiment, the ligand binding domain is an IL-15 domain and ligand binding domain enhancer is an IL-15 domain enhancer.

In embodiments, the covalent complex provided herein including embodiments thereof binds a target ligand expressed on a cell. In embodiments, the target ligand is an interleukin receptor. In embodiments, the interleukin receptor is an IL-15 receptor. In embodiments, the interleukin receptor is an IL-2 receptor. In embodiments, the cell is an immune cell. In embodiments, the immune cell is a T cell. In embodiments, the immune cell is an Nk cell. In embodiments, the cell is a cancer cell. In embodiments, the cell is a breast cancer cell.

In an aspect is provided a nucleic acid including a sequence encoding the covalent complexes provided herein including components thereof.

### Pharmaceutical Compositions

The compositions provided herein include pharmaceutical compositions including the complexes provided herein including embodiments thereof. Thus, in another aspect is provided a pharmaceutical composition including a therapeutically effective amount of a complex as disclosed herein including embodiments thereof and a pharmaceutically acceptable excipient.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Methods of Treatment

The compositions (e.g., multivalent complexes and covalent complexes) provided herein, including embodiments thereof, are contemplated as providing effective treatments for diseases such as cancer (e.g., breast cancer). In embodiments, the cancer is lung cancer, colorectal cancer, melanoma cancer, ovarian cancer, pancreatic cancer, or prostate cancer. In embodiments, the cancer is lung cancer. In embodiments, the cancer is colorectal cancer. In embodiments, the cancer is melanoma cancer. In embodiments, the cancer is ovarian cancer. In embodiments, the cancer is pancreatic cancer. In embodiments, the cancer is prostate cancer.

Thus, in an aspect is provided a method of treating cancer in a subject in need thereof, the method including administering to a subject a therapeutically effective amount of multivalent complex or covalent complex as disclosed herein including embodiments thereof, thereby treating cancer in the subject.

### EMBODIMENTS

Embodiment 1. A multivalent ligand binding complex comprising a first protein dimerizing domain non-covalently bound to a second protein dimerizing domain to form a first ligand binding domain, wherein: (i) said first protein dimerizing domain is covalently bound to a second ligand binding domain through a first chemical linker attached to the N-terminus of said first protein dimerizing domain; and (ii) said first protein dimerizing domain is covalently bound to a second ligand binding domain enhancer through a second chemical linker attached to the C-terminus of said first protein dimerizing domain.

Embodiment 2. A multivalent ligand binding complex comprising a first protein dimerizing domain non-covalently bound to a second protein dimerizing domain to form a first ligand binding domain, wherein: (i) said first protein dimerizing domain is covalently bound to a second ligand binding domain through a first chemical linker attached to the C-terminus of said first protein dimerizing domain; and (ii) said first protein dimerizing domain is covalently bound to a second ligand binding domain enhancer through a second chemical linker attached to the N-terminus of said first protein dimerizing domain.

Embodiment 3. The complex of embodiment 1 or 2, wherein said second ligand binding domain is non-covalently bound to said second ligand binding domain enhancer.

Embodiment 4. The complex of any one of embodiments 1-3, wherein said second ligand binding domain is covalently bound to said second ligand binding domain enhancer through one or more disulfide linkages.

Embodiment 5. The complex of any one of embodiments 1-4, wherein said second ligand binding domain comprises a cysteine at a position corresponding to position 45, 87 or 90 of said second ligand binding domain.

Embodiment 6. The complex of any one of embodiments 1-5, wherein said second ligand binding domain enhancer comprises a cysteine at a position corresponding to position 37, 38, 68 or 67 of said second ligand binding domain enhancer.

Embodiment 7. The complex of any one of embodiments 1-6, wherein said first ligand binding domain is different from said second ligand binding domain.

Embodiment 8. The complex of any one of embodiments 1-7, wherein said complex further comprises a covalent bond connecting said first protein dimerizing domain and said second protein dimerizing domain.

Embodiment 9. The complex of any one of embodiments 1-8, wherein said first protein dimerizing domain is bound to said second protein dimerizing domain.

Embodiment 10. The complex of any one of embodiments 1-9, wherein said first chemical linker is bound to the C-terminus of said second ligand binding domain and said second chemical linker is bound to the N-terminus of said second ligand binding domain enhancer.

Embodiment 11. The complex of any one of embodiments 1-9, wherein said first chemical linker is bound to the C-terminus of said second ligand binding domain enhancer and said second chemical linker is bound to the N-terminus of said second ligand binding domain.

Embodiment 12. The complex of any one of embodiments 1-11, wherein said first protein dimerizing domain comprises a variable light chain domain.

Embodiment 13. The complex of any one of embodiments 1-12, wherein said first protein dimerizing domain comprises a constant light chain domain.

Embodiment 14. The complex of embodiment 13, wherein said constant light chain domain is bound to said second ligand binding domain through said variable light chain domain.

Embodiment 15. The complex of embodiment 13, wherein said constant light chain domain is bound to said second ligand binding domain enhancer through said variable light chain domain.

Embodiment 16. The complex of one of embodiments 1-15, wherein said first protein dimerizing domain is an antibody light chain.

Embodiment 17. The complex of any one of embodiments 1-11, wherein said first protein dimerizing domain comprises a variable heavy chain domain.

Embodiment 18. The complex of embodiment 17, wherein said first protein dimerizing domain comprises a constant heavy chain domain.

Embodiment 19. The complex of embodiment 18, wherein said constant heavy chain domain is bound to said second ligand binding domain through said variable heavy chain domain.

Embodiment 20. The complex of embodiment 18, wherein said constant heavy chain domain is bound to said second ligand binding domain enhancer through said variable heavy chain domain.

Embodiment 21. The complex of one of embodiments 17-20, wherein said first protein dimerizing domain is an antibody heavy chain.

Embodiment 22. The complex of any one of embodiments 1-16, wherein said second protein dimerizing domain comprises a constant heavy chain domain.

Embodiment 23. The complex of embodiment 22, wherein said second protein dimerizing domain comprises a variable heavy chain domain.

Embodiment 24. The complex of embodiment 23, wherein said second protein dimerizing domain is an antibody heavy chain.

Embodiment 25. The complex of any one of embodiments 17-21, wherein said second protein dimerizing domain comprises a constant light chain domain.

Embodiment 26. The complex of embodiment 25, wherein said second protein dimerizing domain comprises a variable light chain domain.

Embodiment 27. The complex of embodiment 23, wherein said second protein dimerizing domain is an antibody light chain.

Embodiment 28. The complex of any one of embodiments 1-27, wherein said first ligand binding domain is a Fab domain.

Embodiment 29. The complex of any one of embodiments 1-28, wherein said first protein dimerizing domain is bound to an Fc domain through a third chemical linker.

Embodiment 30. The complex of any one of embodiments 1-28, wherein said second protein dimerizing domain is bound to an Fc domain through a third chemical linker.

Embodiment 31. The complex of any one of embodiments 1-30, wherein said first ligand binding domain is an anti PDL-1 binding domain, an anti L1 CAM binding domain, an anti-EGFR binding domain or an anti-CEA binding domain.

Embodiment 32. The complex of any one of embodiments 1-31, wherein said second ligand binding domain is a chemokine domain.

Embodiment 33. The complex of any one of embodiments 1-32, wherein said second ligand binding domain is an interleukin domain.

Embodiment 34. The complex of any one of embodiments 1-33, wherein said second ligand binding domain is an IL-2 domain, an IL-4 domain, an IL-7 domain, an IL-9 domain, an IL-15 domain, an IL-21 domain or a thymic stromal lymphopoietin (TSLP) domain.

Embodiment 35. The complex of any one of embodiments 1-34, wherein said second ligand binding domain enhancer is a chemokine domain enhancer.

Embodiment 36. The complex of any one of embodiments 1-35, wherein said second ligand binding domain enhancer is an interleukin domain enhancer.

Embodiment 37. The complex of any one of embodiments 1-36, wherein said second ligand binding domain enhancer comprises a sushi domain.

Embodiment 38. The complex of any one of embodiments 1-36, wherein said second ligand binding domain enhancer is an IL-2 domain enhancer, an IL-4 domain enhancer, an IL-7 domain enhancer, an IL-9 domain enhancer, an IL-15 domain enhancer, an IL-21 domain enhancer or a thymic stromal lymphopoietin (TSLP) domain enhancer.

Embodiment 39. The complex of any one of embodiments 1-38, wherein said first chemical linker is a peptidyl linker.

Embodiment 40. The complex of any one of embodiments 1-39, wherein said second chemical linker is a peptidyl linker.

Embodiment 41. The complex of any one of embodiments 1-40, wherein said first chemical linker and said second chemical linker are independently a covalent linker or a non-covalent linker.

Embodiment 42. The complex of any one of embodiments 1-40, wherein said first chemical linker and said second chemical linker are independently a cleavable peptide linker.

Embodiment 43. The complex of any one of embodiments 1-42, wherein said first chemical linker and said second chemical linker are independently an enzymatically cleavable linker.

Embodiment 44. The complex of any one of embodiments 1-43, wherein said first chemical linker and said second chemical linker are independently a protease cleavable linker.

Embodiment 45. The complex of any one of embodiments 1-44, wherein said first chemical linker and said second chemical linker are independently a tumor-associated protease cleavable linker.

Embodiment 46. The complex of any one of embodiments 1-45, wherein said first chemical linker and said second chemical linker independently have a length of about 0 to about 15 amino acid residues.

Embodiment 47. The complex of any one of embodiments 1-46, wherein said first chemical linker and said second chemical linker independently comprise a BSA binding moiety.

Embodiment 48. A pharmaceutical composition comprising a complex of any one of embodiments 1-47 and a pharmaceutically acceptable excipient.

### EXAMPLES

### Example 1

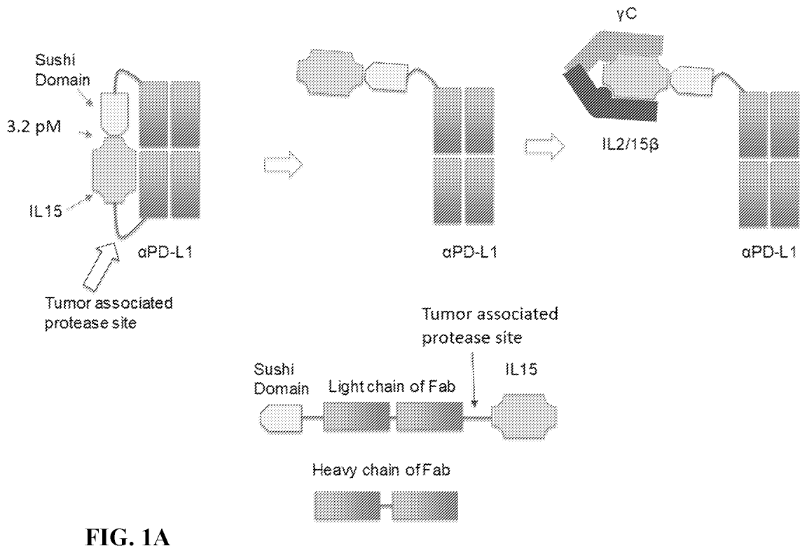

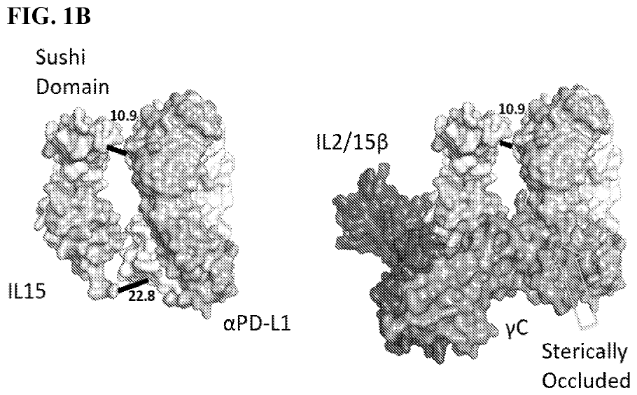

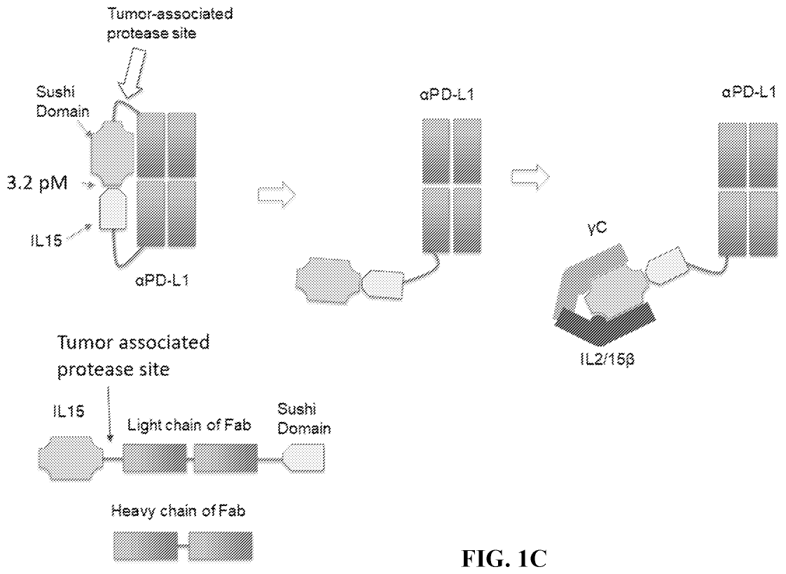

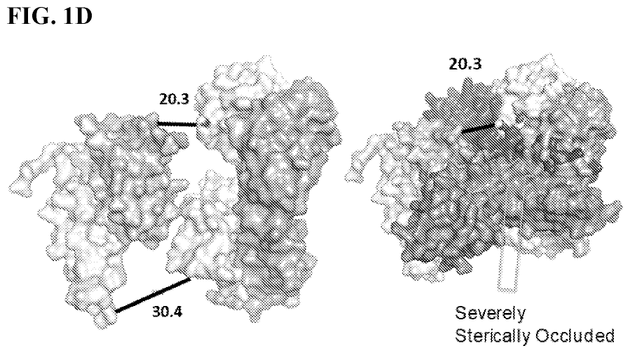

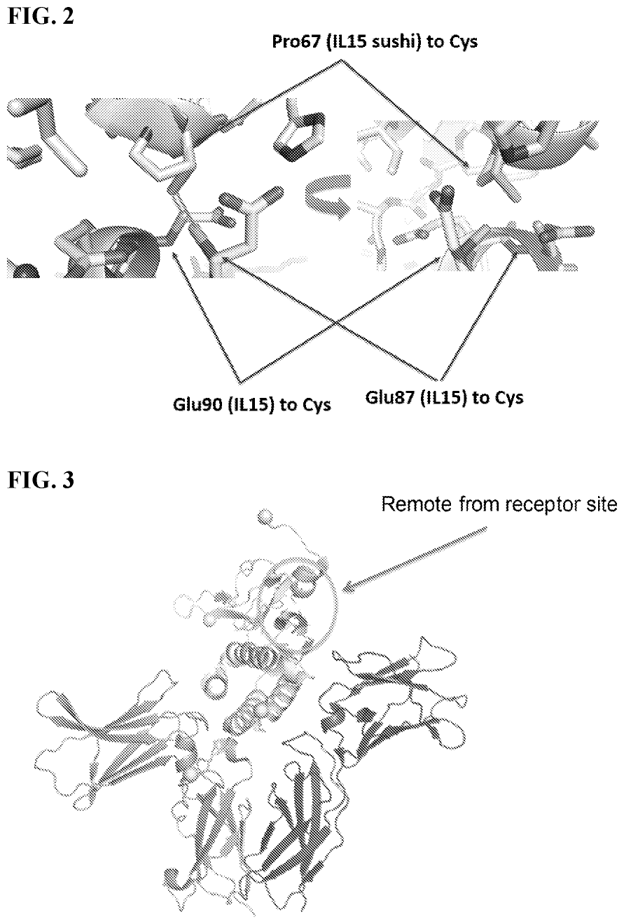

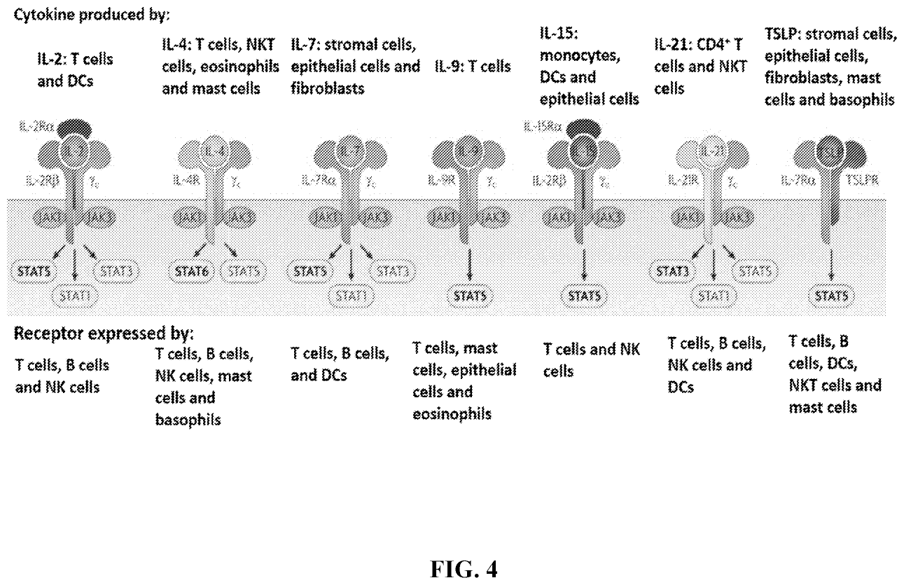

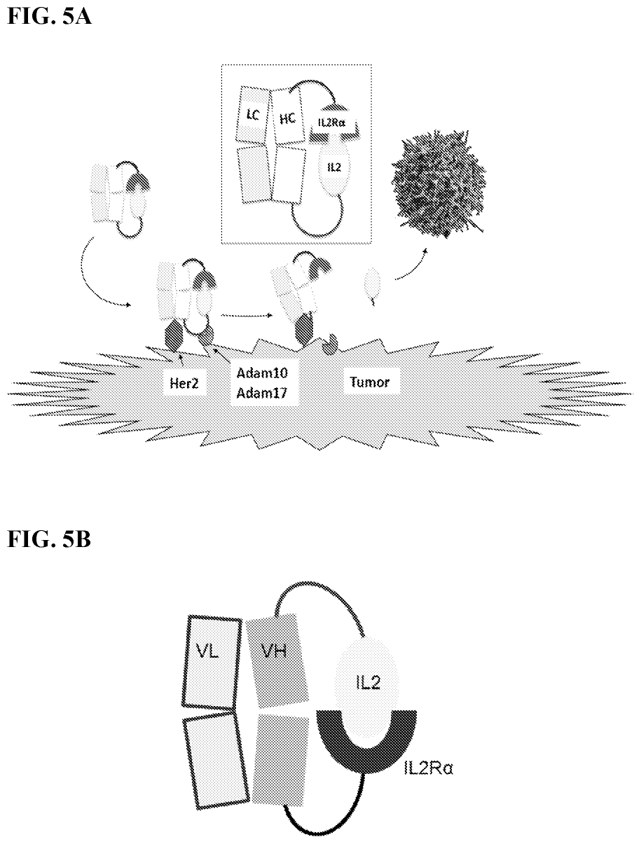

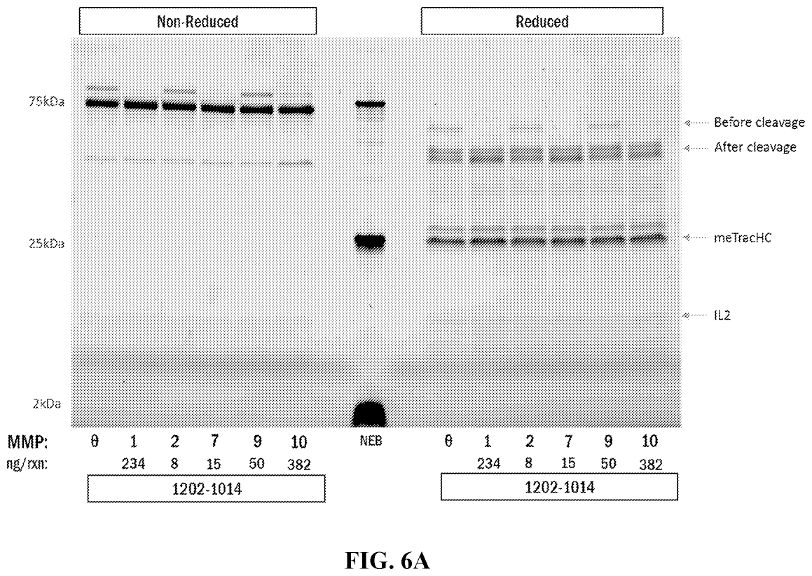

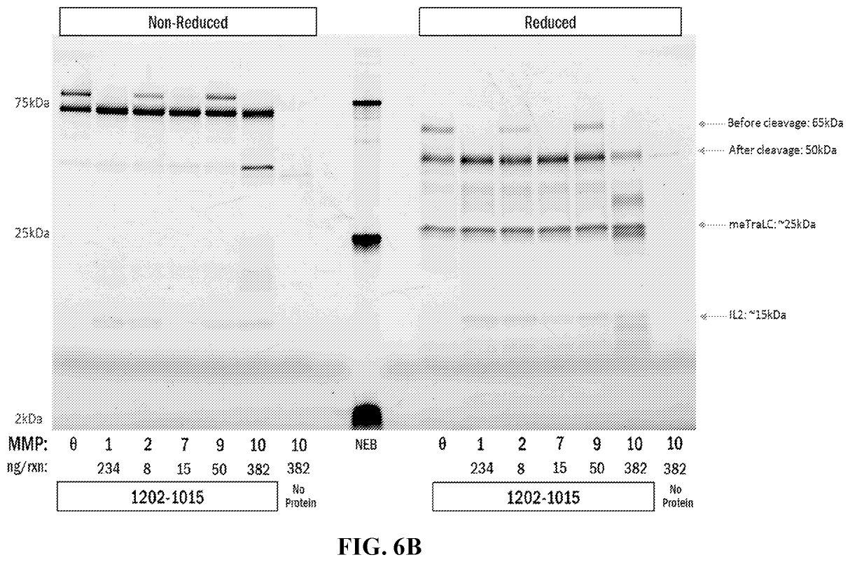

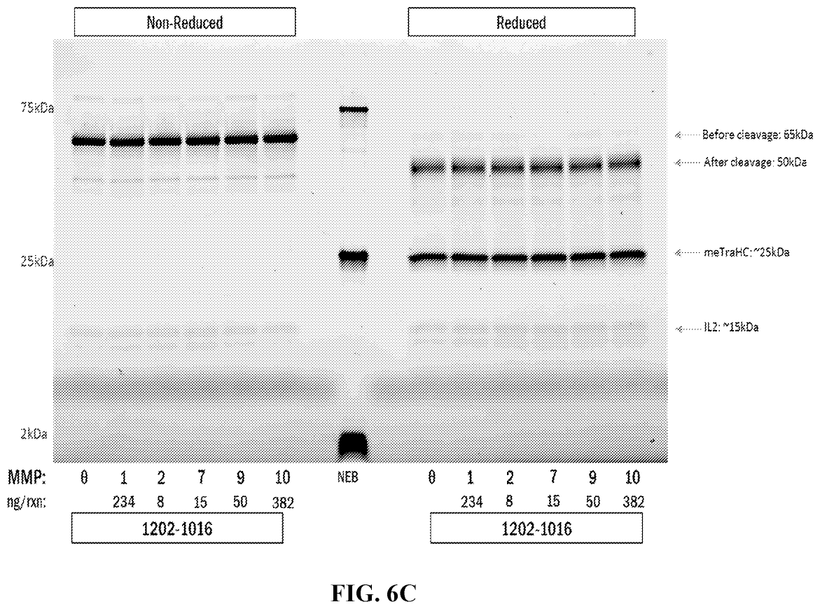

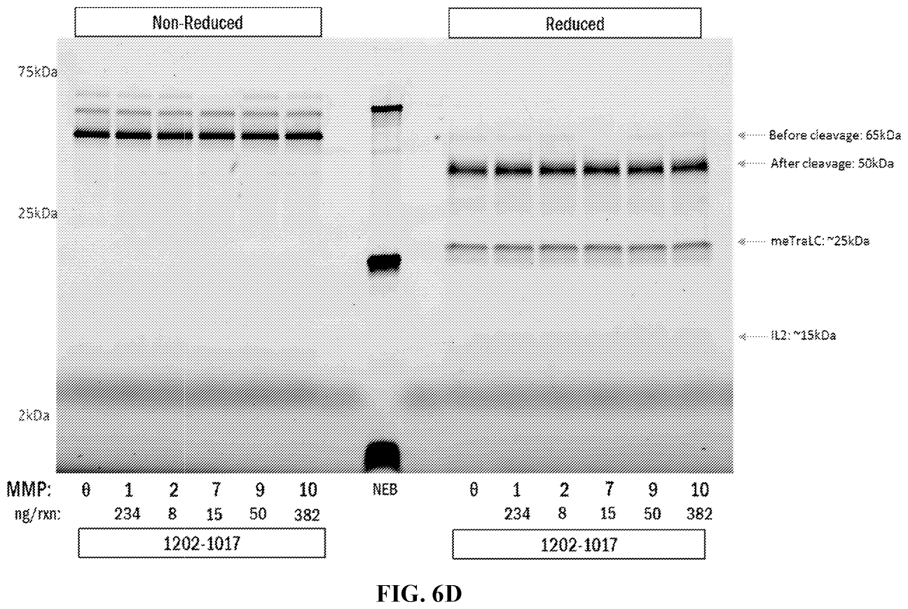

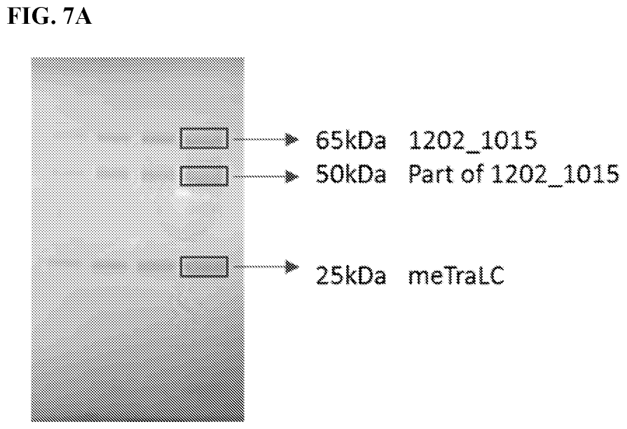

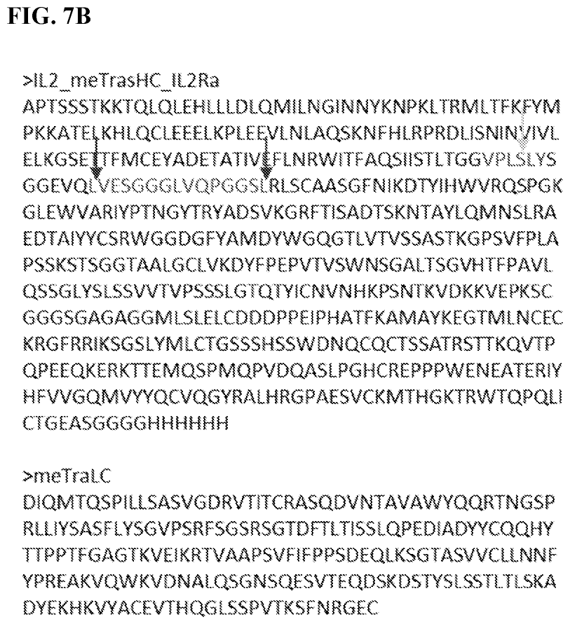

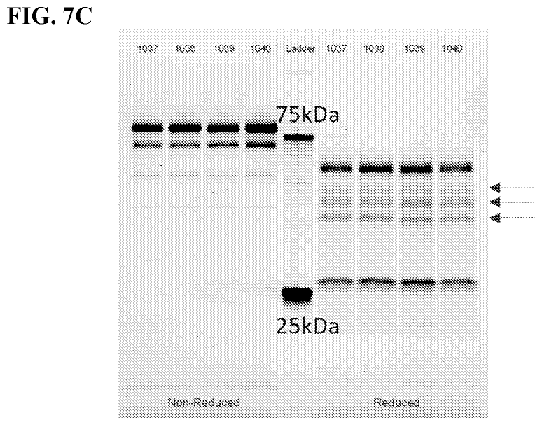

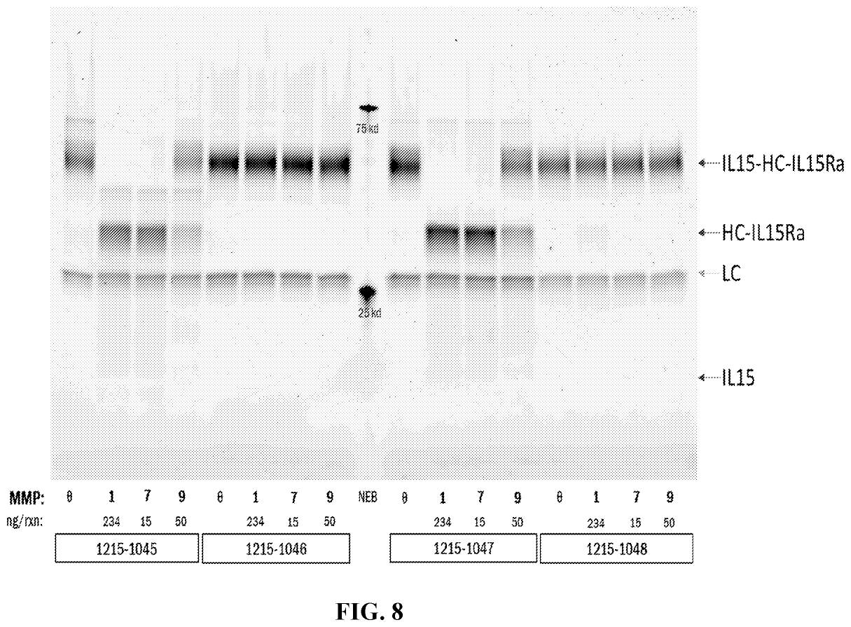

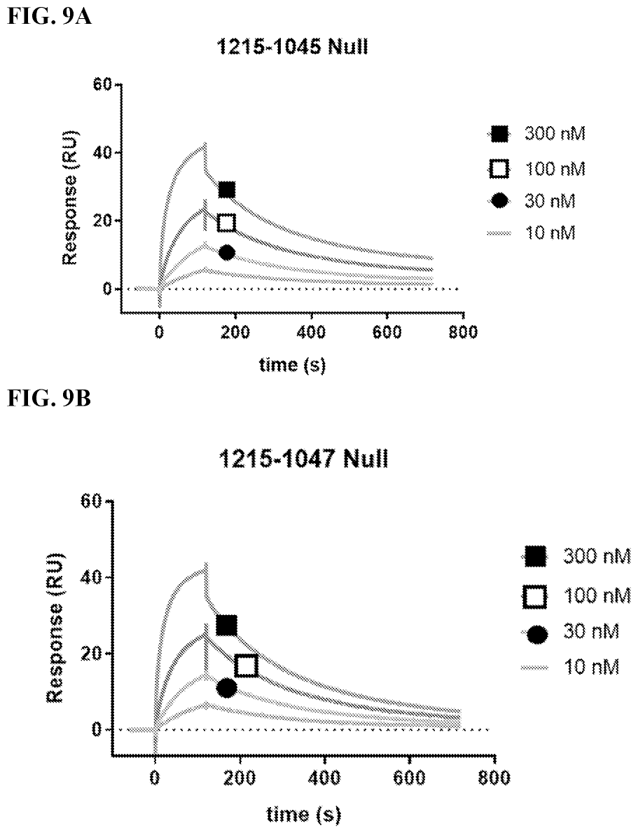

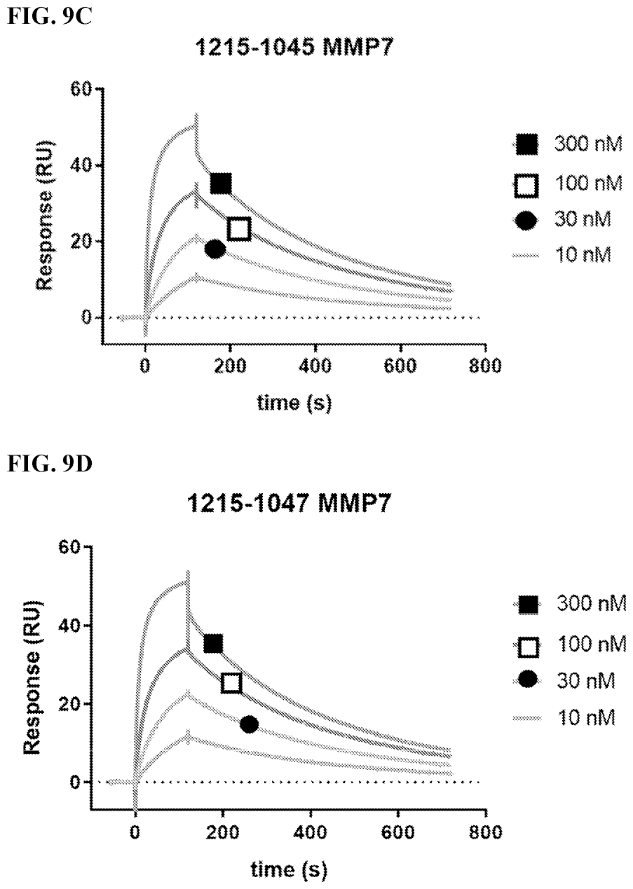

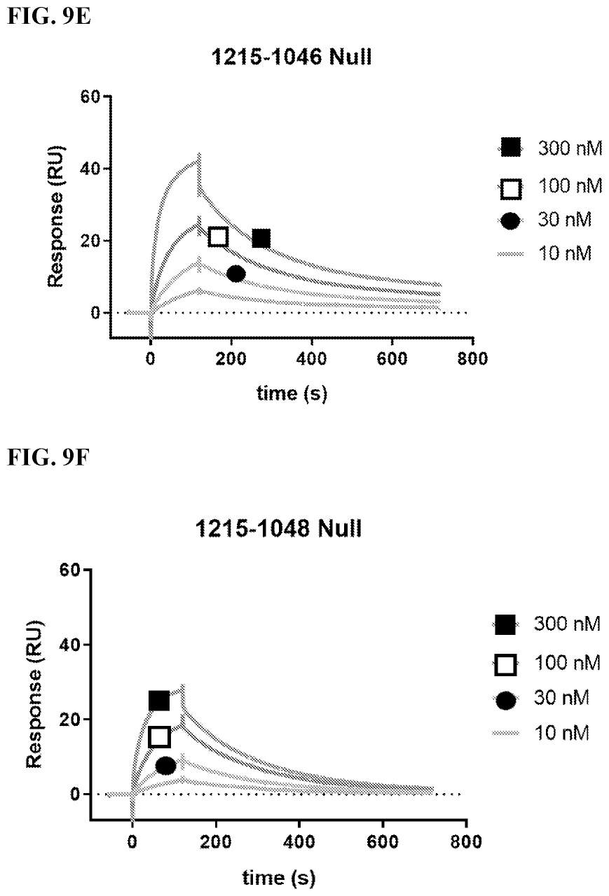

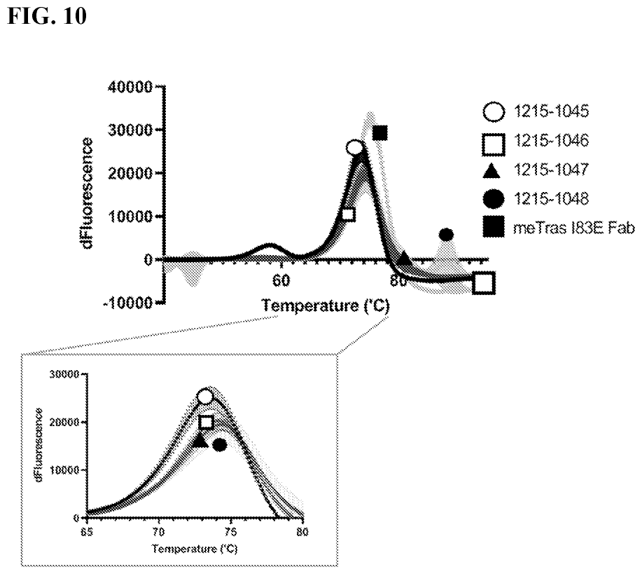

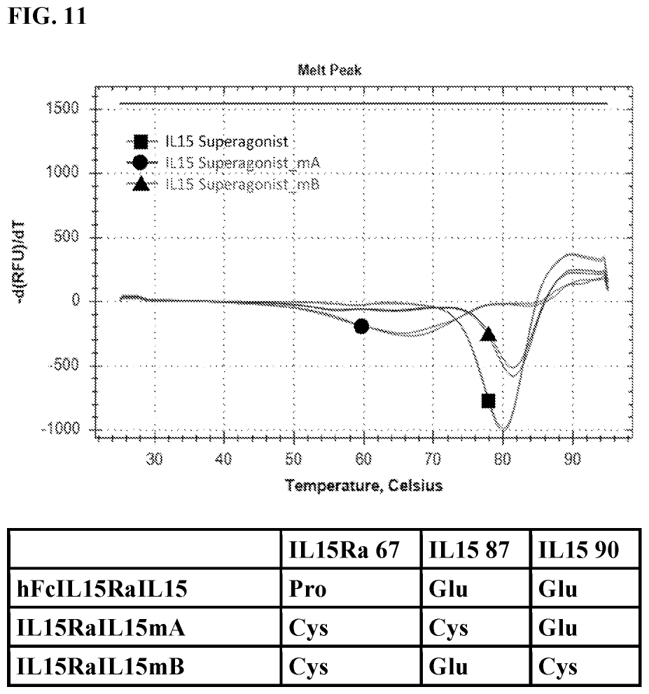

Two different configurations on one Fab chain (FIGS. 1A-1D). Note the other Fab chain is also amenable to this. We can fuse the Fc to either the light or heavy chain depending on the aklusion if needed. The construct is going to be pretty large and may not require the Fc.

Alternatively, we can add an albumin tag at the C-terminus (to improve serum half-life). In addition, we are relying on the ultra high affinity of the sushi/IL-15 interaction. We may build in disulfide bonds (there are several positions that could support its formation (including residue 37 on the sushi domain to residue 45 on IL-15; Residue 38 on sushi to residue 45 on IL-15; Residue 68 on sushi to residue 90 on IL-15; Residue 67 on sushi to residue 90 on IL-15 (favorite); Residue 67 on sushi to residue 87 on IL-15 (another favorite) (see next figure). Non limiting examples of first ligand binding domains are PDL1, L1CAM, EGFRv3, CEA, Mesothelin, CDH6, Her2, Her3, Ax1, antibodies specific to MHC molecules that are bound to intracellular peptides associated with cancer (e.g., kRas, IDH2).

### Example 2: IL-15-Fab/MAB Designs

IL-15 is an important cytokine that activates T cells and NK cells and does not induce apoptosis. IL-15 binds to the IL2/15β receptor and γC receptor. IL-15 also binds to the IL-15a receptor, a sushi domain protein that interacts with IL-15 with 3.2 pM affinity and dramatically enhances the IL-15 affinity to IL2/15β receptor and γC receptor. In order to improve the tissue specificity, IL-15 is typically fused to a targeting moiety (e.g. the C-terminus of an antibody). In addition, IL-15 has been fused to the sushi domain to improve its expression and affinity for the IL2/IL-15β/γC receptor. A significant concern with this approach is on-target, off-tissue toxicities. IL-15 can be fused to the sushi domain to improve its expression and affinity for the IL2/IL-15β/γC receptor, however a significant concern with this approach is on-target, off-tissue toxicities. To mitigate these concerns, we visually examined the crystal structure of the IL-15 complex. We observed that the N- and C-termini of the IL-15 and the sushi domain were positioned on the opposite side of the complex. This circumstance suggests that we could fuse the sushi domain to one termini of the light or heavy chain and fuse IL-15 to the other termini. By placing the IL-15/sushi complex next to the light or heavy chain of the Fab and adjusting the IL-15/sushi complex such that the N- and C-termini were close to the C- or N-termini of the Fab, it was clear that the fusion of the termini could sterically occlude the IL2/15β receptor or γC receptor. Placing a tumor activated sequence between the IL-15 and the Fab will be used to selectively activate the fusion at the site of disease. This will be used for IL2 as well.

### Example 3: Occluded IL-15-Fab Variants

We will produce a series of novel tumor-activated, IL-15/sushi-Fab constructs for potential use in animal studies. Key steps are: production of 40 Fab variants based on PDL1; characterization of IL2/15β and γC receptor aklusion; characterization of activation through proteolysis of common substrate; substitution of additional protease substrates and their characterization; in vitro characterization (differentiation of monocytes, activation of JAK/STAT pathways) as well as animal models (using surrogate murine α-PDL1). In addition, we will also characterize the N72D mutation. Using the complexes provided herein we will be providing, inter alia, a tumor-activated IL-15 biologic ready for pre-clinical development.

Synthesis of base DNA (IL-15-Fab LC-sushi, IL-15-Fab HC-sushi, sushi-Fab LC-IL-15 and sushi-Fab HC-IL-15) will be performed. Production of base molecules will involve Maxi-prep (Qiagen) large scale DNA purification to ensure high yield of high quality of DNA. Expression of antibody will be performed in expiCHO cells. Typical volumes range from 100-200 ml with anticipated yield of 10-100 mg (antibody dependent). Protein will be purified to homogeneity and its purity will be confirmed by SDS PAGE.

Each base molecule will be characterized by mass spec, SPR and DSF. To this end, we will test the uncleaved and cleaved molecules.

We will establish the parameters for ELISA assays as well as measure the binding of the occluded and activated molecules using analytical cytometry (i.e. FACS). The linker between IL-15 and the Fab will be systematically explored (shorter and longer) and the composition of the substrates will be optimized for activation properties (40 variants). To achieve this, we will combine modeling and ELISA assays to ensure full activation.

We will make non-cleavable versions as well as ALT-803 and IL-15SA-IL-15RaSU-Fc to bench mark. We will use CT26 and 4T1 syngeneic tumor models as well. First, we will dose tumor bearing and non-tumor bearing animals. Serum will be collected at 1, 2, 3, and 5 days and IFN-g, TNFa, IL6 and IL10 concentrations will be determined. Next, we will characterize the status of a number of lymphocytes including NK cells (CD49b+), Tregs (CD4+/FOXP3+) T cells, B cells MDSCs as a function of time (e.g., daily over a week).

We will establish the parameters for ELISA assays as well as measure the binding of the occluded and activated molecules using analytical cytometry (i.e. FACS). The linker between IL-15 and the Fab will be systematically explored (shorter and longer) and the composition of the substrates will be optimized for activation properties (40 variants). To achieve this, we will combine modeling and ELISA assays to ensure full activation.

We will make non-cleavable versions as well as ALT-803 and IL-15SA-IL-15RaSU-Fc to bench mark. We will use CT26 and 4T1 syngeneic tumor models as well. First, we will dose tumor bearing and non-tumor bearing animals. Serum will be collected at 1, 2, 3, and 5 days and IFN-g, TNFa, IL6 and IL10 concentrations will be determined. Next, we will characterize the status of a number of lymphocytes including NK cells (CD49b+), Tregs (CD4+/FOXP3+) T cells, B cells MDSCs as a function of time (e.g., daily over a week).

### REFERENCES

Kim P S, Kwilas A R, Xu W, Alter S, Jeng E K, Wong H C, Schlom J, Hodge J W. IL-15 superagonist/IL-15RαSushi-Fc fusion complex (IL-15SA/IL-15RαSu-Fc; ALT-803) markedly enhances specific subpopulations of NK and memory CD8+ T cells, and mediates potent anti-tumor activity against murine breast and colon carcinomas. Oncotarget. 2016 Mar. 29; 7(13):16130-45.

### TABLES

TABLE 1
Linker sequences of IL2-Fab complexes

| | | | SEQ | | SEQ | Cleavage |
|---|---|---|---|---|---|---|
| | | Linker 1 | ID NO. | Linker 2 | ID NO. | site |
| IL2_linker1_LC | 1202_1014 | GGVPLSLYSGG | 1 | GGASGSGAGAG | 2 | MMP |
| Fab_linker2_IL2Ra | | | | | | |
| and HC Fab | | | | | | |
| IL2_linker1_HC | 1202_1015 | GGVPLSLYSGG | 3 | GGGSGAGAG | 4 | MMP |
| Fab_linker2_IL2Ra | | | | | | |
| and LC Fab | | | | | | |
| IL2Ra_linker1_LC | 1202_1016 | GGGSGG | 5 | GGGGVPLSLYSGG | 6 | MMP |
| Fab_linker2_IL2 | | | | | | |
| and HC Fab | | | | | | |
| IL2Ra_linker1_HC | 1202_1017 | GGGSGG | 7 | GGGGVPLSLYSGG | 8 | MMP |
| Fab_linker2_IL2 | | | | | | |
| and LC Fab | | | | | | |

TABLE 2
Fragments of IL2-Fab complexes including linker and adjacent sequences
Mutations for avoiding potential non-MMP dependent
cleavage are in bold, underlined text.

| | |
|---|---|
| Linker Sequence | SEQ ID NO. |

IL2_linker1_HC Fab_linker2_IL2Ra and LC Fab

| | | |
|---|---|---|
| 1202_1015 | GGVPLSLYSGGEVQLVESGGGLVQPGGSLRL | 12 |
| 1202_1037 | GGVPLSLYSGGEVQLVESGGGLVQPGDSLRL | 13 |

IL2_linker1_HC Fab_linker2_IL2Ra and LC Fab

| | | |
|---|---|---|
| 1202_1038 | GGVPLSLYSGGEVQLVESGGGLVQPGESLRL | 14 |
| 1202_1039 | GGVPLSLYSGGAVQLVESGGGLVQPGGSLRL | 15 |
| 1202_1040 | GGVPLSLYSGGSVQLVESGGGLVQPGGSLRL | 16 |

TABLE 3
Linker sequences of IL-15-Fab complexes.

| | | | | | |
|---|---|---|---|---|---|
| Linker1 | SEQ ID NO. | Linker2 | SEQ ID NO. | Cleavage site | Note |

IL-15_linker1_HC Fab_linker2_IL-15Ra and LC Fab

| | | | | | | |
|---|---|---|---|---|---|---|
| 1215_1045 | GGVPLSLYSGG | 17 | GGGSGAGAG | 18 | MMP | |
| 1215_1046 | GGSPSGASGS | 19 | GGGSGAGAG | 20 | Non- | |
| | | | | | cleavable | |
| 1215_1047 | GGVPLSLYSGG | 21 | GGGSGAGAG | 22 | MMP | Cys mutations |
| | | | | | | on cytokines |
| | | | | | | (IL-15Ra- |
| | | | | | | P67C, IL-15-E90C) |
| 1215_1048 | GGSPSGASGS | 23 | GGGSGAGAG | 24 | Non- | Cys mutations |
| | | | | | cleavable | on cytokines |
| | | | | | | IL-15Ra-P67C, |
| | | | | | | IL-15-E90C) |
| 1215_1050 | TIVPLSLYWN | 25 | TFPVLV | 26 | MMP | Low |
| | | | | | | production |
| | | | | | | (<1 mg/L) |

IL-15_linker1_HC Fab_linker2_IL-15Ra and LC Fab

| | | | | | | |
|---|---|---|---|---|---|---|
| 1215_1051 | VVPLSLYWN | 27 | TFPVLV | 28 | MMP | Low |
| | | | | | | production |
| | | | | | | (<1 mg/L) |
| 1215_1053 | ARLAELNA | 29 | VPLSLY | 30 | MMP | Low |
| | | | | | | production |
| | | | | | | (<1 mg/L) |
| 1215_1054 | IVPLSLY | 31 | TFPVLV | 32 | MMP | Low |
| | | | | | | production |
| | | | | | | (<1 mg/L) |
| 1215_1059 | VVPLSLYWN | 33 | VVPLSLYWN | 34 | MMP | Dual cleavage |
| | | | | | | sites, |
| | | | | | | Expression |
| | | | | | | failed |

TABLE 4
Linker sequences of IL-15-Fab complexes with a different conformation.
IL-15Ra_linker1_LC Fab_linker2_IL-15 and HC Fab

| | | | | | Cleavage | |
|---|---|---|---|---|---|---|
| | Linker 1 | SEQ ID NO. | Linker 2 | SEQ ID NO. | site | Note |
| 1215_1055 | VPLSLYFT | 35 | IQEARERWNF | 36 | MMP | Expression |
| | | | | | | failed |
| 1215_1056 | TFPVLVRT | 37 | TLVPLSLYWK | 38 | MMP | Not |
| | | | | | | produced yet |

TABLE 5
Kinetics of IL-15-Fab complexes binding
to IL-15Rβ-Fc as determined by SPR.

| | ka | kd | KD | Chi² | U- |
|---|---|---|---|---|---|
| Fc binding | (1/Ms) | (1/s) | (M) | (RU²) | value |
| 1215-1045 null | 1.17E+05 | 2.56E−03 | 2.19E−08 | 1.13 | 2 |
| 1215-1045 mmp7 | 2.51E+05 | 2.69E−03 | 1.07E−08 | 1.54 | 2 |
| 1215-1046 null | 1.50E+05 | 3.08E−03 | 2.05E−08 | 1.32 | 2 |
| 1215-1047 null | 1.64E+05 | 3.75E−03 | 2.29E−08 | 1 | 2 |
| 1215-1047 mmp7 | 2.81E+05 | 2.84E−03 | 1.01E−08 | 1.56 | 2 |
| 1215-1048 null | 1.89E+05 | 6.32E−03 | 3.34E−08 | 0.331 | 2 |

TABLE 6
Melting temperatures of various IL2-Fab complexes,
as determined by differential scanning fluorimetry.

| | Tm (′C.) |
|---|---|
| 1215-1045 | 71.96 +/− 0.037 |
| 1215-1046 | 73.32 +/− 0.047 |
| 1215-1047 | 73.35 +/− 0.146 |

INFORMAL SEQUENCE LISTING

1202_1014 Linker 1 (SEQ ID NO: 1):
GGVPLSLYSGG
1202_1014 Linker 2 (SEQ ID NO: 2):
GGASGSGAGAG
1202_1015 Linker 1 (SEQ ID NO: 3):
GGVPLSLYSGG
1202_1015 Linker 2 (SEQ ID NO: 4):
GGGSGAGAG
1202_1016 Linker 1 (SEQ ID NO: 5):
GGGSGG
1202_1016 Linker 2 (SEQ ID NO: 6):
GGGGVPLSLYSGG
1202_1017 Linker 1 (SEQ ID NO: 7):
GGGSGG
1202_1017 Linker 2 (SEQ ID NO: 8):
GGGGVPLSLYSGG
IL2_meTrasHC_IL2Ra (SEQ ID NO: 9)
APTSSSTKKTQLQLEHLLLDLQMILNGINNYKNPKLTRMLTFKFYMPKKATELKHLQCLEEELKPLEEVL
NLAQSKNFHLRPRDLISNINVIVLELKGSETTFMCEYADETATIVEFLNRWITFAQSIISTLTGGVPLSL
YSGGEVQLVESGGGLVQPGGSLRLSCAASGFNIKDTYIHWVRQSPGKGLEWVARIYPTNGYTRYADSVKG
RFTISADTSKNTAYLQMNSLRAEDTAIYYCSRWGGDGFYAMDYWGQGTLVTVSSASTKGPSVFPLAPSSK
STSGGTAALGCLVKDYFPEPVTVSWNSGALTSGVHTFPAVLQSSGLYSLSSVVTVPSSSLGTQTYICNVN
HKPSNTKVDKKVEPKSCGGGSGAGAGGMLSLELCDDDPPEIPHATFKAMAYKEGTMLNCECKRGFRRIKS
GSLYMLCTGSSSHSSWDNQCQCTSSATRSTTKQVTPQPEEQKERKTTEMCISPMQPVDQASLPGHCREPP
PWENEATERIYHFVVGQMVYYQCVQGYRALHRGPAESVCKMTHGKTRWTQPQLICTGEASGGGGHHHHHH
meTraLC (SEQ ID NO: 10)
DIQMTQSPILLSASVGDRVTITCRASQDVNTAVAWYQQRTNGSPRLLIYSASFLYSGVPSRFSGSRSGTD
FTLTISSLQPEDIADYYCQQHYTTPPTFGAGTKVEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSFN
RGEC
Protease site (SEQ ID NO: 11)
EVQLVESG
1202_1015 (SEQ ID NO: 12)
GGVPLSLYSGGEVQLVESGGGLVQPGGSLRL
1202_1037 (SEQ ID NO: 13)
GGVPLSLYSGGEVQLVESGGGLVQPGDSLRL
1202_1038 (SEQ ID NO: 14)
GGVPLSLYSGGEVQLVESGGGLVQPGESLRL
1202_1039 (SEQ ID NO: 15)
GGVPLSLYSGGAVQLVESGGGLVQPGGSLRL
1202_1040 (SEQ ID NO: 16)
GGVPLSLYSGGSVQLVESGGGLVQPGGSLRL
1215_1045 Linker 1 (SEQ ID NO: 17)
GGVPLSLYSGG
1215_1045 Linker 2 (SEQ ID NO: 18)
GGGSGAGAG
1215_1046 Linker 1 (SEQ ID NO: 19)
GGSPSGASGS
1215_1046 Linker 2 (SEQ ID NO: 20)
GGGSGAGAG
1215_1047 Linker 1 ((SEQ ID NO: 21)
GGVPLSLYSGG
1215_1047 Linker 2 (SEQ ID NO: 22)
GGGSGAGAG
1215_1048 Linker 1 (SEQ ID NO: 23)
GGSPSGASGS
1215_1048 Linker 2 (SEQ ID NO: 24)
GGGSGAGAG
1215_1050 Linker 1 (SEQ ID NO: 25)
TIVPLSLYWN
1215_1050 Linker 2 (SEQ ID NO: 26)
TFPVLV
1215_1051 Linker 1 (SEQ ID NO: 27)
VVPLSLYWN
1215_1051 Linker 2 (SEQ ID NO: 28)
TFPVLV
1215_1053 Linker 1 (SEQ ID NO: 29)
ARLAELNA
1215_1053 Linker 2 (SEQ ID NO: 30)
VPLSLY
1215_1054 Linker 1 (SEQ ID NO: 31)
IVPLSLY
1215_1054 Linker 2 (SEQ ID NO: 32)
TFPVLV
1215_1059 Linker 1 (SEQ ID NO: 33)
VVPLSLYWN
1215_1059 Linker 2 (SEQ ID NO: 34)
VVPLSLYWN
1215_1055 Linker 1 (SEQ ID NO: 35)
VPLSLYFT
1215_1055 Linker 2 (SEQ ID NO: 36)
IQEARERWNF
1215_1056 Linker 1 (SEQ ID NO: 37)
TFPVLVRT
1215_1056 Linker 2 (SEQ ID NO: 38)
TLVPLSLYWK
meTrasHC (SEQ ID NO: 39)
EVQLVESGGGLVQPGGSLRLSCAASGFNIKDTYIHWVRQSPGKGLEWVARIYPTNGYTRYADSVKGRFTI
SADTSKNTAYLQMNSLRAEDTAIYYCSRWGGDGFYAMDYWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKPS
NTKVDKKVEPKSC
IL-2 (SEQ ID NO: 40)
APTSSSTKKTQLQLEHLLLDLQMILNGINNYKNPKLTRMLTFKFYMPKKATELKHLQCLEEELKPLEEVL
NLAQSKNFHLRPRDLISNINVIVLELKGSETTFMCEYADETATIVEFLNRWITFAQSIISTLT
Interleukin-2 receptor alpha (SEQ ID NO: 41)
SLELCDDDPPEIPHATFKAMAYKEGTMLNCECKRGFRRIKSGSLYMLCTGSSSHSSWDNQCQCTSSATRS
TTKQVTPQPEEQKERKTTEMQSPMQPVDQASLPGHCREPPPWENEATERIYHFVVGQMVYYQCVQGYRAL
HRGPAESVCKMTHGKTRWTQPQLICTGE
IL-15 (SEQ ID NO: 42)
NWVNVISDLKKIEDLIQSMHIDATLYTESDVHPSCKVTAMKCFLLELQVISLESGDASIHDTVENLIILA
NNSLSSNGNVTESGCKECEELEEKNIKEFLQSFVHIVQMFINTS
Interleukin-15 receptor alpha (SEQ ID NO: 43)
ITCPPPMSVEHADIWVKSYSLYSRERYICNSGFKRKAGTSSLTECVLNKATNVAHWTTPSLKCIRDPALV
HQRPAPPS
Interleukin-15 receptor alpha (IL-15Ra-P67C) (SEQ ID NO: 44)
ITCPPPMSVEHADIWVKSYSLYSRERYICNSGFKRKAGTSSLTECVLNKATNVAHWTTPSLKCIRDCALV
HQRPAPPS
IL-15 (IL-15-E90C) (SEQ ID NO: 45)
NWVNVISDLKKIEDLIQSMHIDATLYTESDVHPSCKVTAMKCFLLELQVISLESGDASIHDTVENLIILA
NNSLSSNGNVTESGCKECCELEEKNIKEFLQSFVHIVQMFINTS

What is claimed is:

1. A multivalent ligand binding complex comprising a first protein dimerizing domain non-covalently bound to a second protein dimerizing domain to form a first ligand binding domain, wherein:
(i) said first protein dimerizing domain is covalently bound to a second ligand binding domain through a first chemical linker attached to the N-terminus of said first protein dimerizing domain; and
(ii) said first protein dimerizing domain is covalently bound to a second ligand binding domain enhancer through a second chemical linker attached to the C-terminus of said first protein dimerizing domain.

2. A multivalent ligand binding complex comprising a first protein dimerizing domain non-covalently bound to a second protein dimerizing domain to form a first ligand binding domain, wherein:
(i) said first protein dimerizing domain is covalently bound to a second ligand binding domain through a first chemical linker attached to the C-terminus of said first protein dimerizing domain; and
(ii) said first protein dimerizing domain is covalently bound to a second ligand binding domain enhancer through a second chemical linker attached to the N-terminus of said first protein dimerizing domain.

3. The complex of claim 1 or 2, wherein said second ligand binding domain is non-covalently bound to said second ligand binding domain enhancer.

4. The complex of claim 1 or 2, wherein said second ligand binding domain is covalently bound to said second ligand binding domain enhancer through one or more disulfide linkages.

5. The complex of claim 1 or 2, wherein said second ligand binding domain comprises a cysteine at a position corresponding to position 45, 87 or 90 of said second ligand binding domain.

6. The complex of claim 1 or 2, wherein said second ligand binding domain enhancer comprises a cysteine at a position corresponding to position 37, 38, 68 or 67 of said second ligand binding domain enhancer.

7. The complex of claim 1 or 2, wherein said first ligand binding domain is different from said second ligand binding domain.

8. The complex of claim 1 or 2, wherein said complex further comprises a covalent bond connecting said first protein dimerizing domain and said second protein dimerizing domain.

9. The complex of claim 1 or 2, wherein said first protein dimerizing domain is bound to said second protein dimerizing domain.

10. The complex of claim 1 or 2, wherein said first chemical linker is bound to the C-terminus of said second ligand binding domain and said second chemical linker is bound to the N-terminus of said second ligand binding domain enhancer.

11. The complex of claim 1 or 2, wherein said first chemical linker is bound to the C-terminus of said second ligand binding domain enhancer and said second chemical linker is bound to the N-terminus of said second ligand binding domain.

12. The complex of claim 1 or 2, wherein said first protein dimerizing domain comprises a variable light chain domain.

13. The complex of claim 1 or 2, wherein said first protein dimerizing domain comprises a constant light chain domain.

14. The complex of claim 13, wherein said constant light chain domain is bound to said second ligand binding domain through said variable light chain domain.

15. The complex of claim 13, wherein said constant light chain domain is bound to said second ligand binding domain enhancer through said variable light chain domain.

16. The complex of claim 1 or 2, wherein said first protein dimerizing domain is an antibody light chain.

17. The complex of claim 1 or 2, wherein said first protein dimerizing domain comprises a variable heavy chain domain.

18. The complex of claim 17, wherein said first protein dimerizing domain comprises a constant heavy chain domain.

19. The complex of claim 18, wherein said constant heavy chain domain is bound to said second ligand binding domain through said variable heavy chain domain.

20. The complex of claim 18, wherein said constant heavy chain domain is bound to said second ligand binding domain enhancer through said variable heavy chain domain.

21. The complex of claim 1 or 2, wherein said first protein dimerizing domain is an antibody heavy chain.

22. The complex of claim 1 or 2, wherein said second protein dimerizing domain comprises a constant heavy chain domain.

23. The complex of claim 22, wherein said second protein dimerizing domain comprises a variable heavy chain domain.

24. The complex of claim 23, wherein said second protein dimerizing domain is an antibody heavy chain.

25. The complex of claim 1 or 2, wherein said second protein dimerizing domain comprises a constant light chain domain.

26. The complex of claim 25, wherein said second protein dimerizing domain comprises a variable light chain domain.

27. The complex of claim 23, wherein said second protein dimerizing domain is an antibody light chain.

28. The complex of claim 1 or 2, wherein said first ligand binding domain is a Fab domain.

29. The complex of claim 1 or 2, wherein said first protein dimerizing domain is bound to an Fc domain through a third chemical linker.

30. The complex of claim 1 or 2, wherein said second protein dimerizing domain is bound to an Fc domain through a third chemical linker.

31. The complex of claim 1 or 2, wherein said first ligand binding domain is an anti PDL-1 binding domain, an anti L1 CAM binding domain, an anti-EGFR binding domain or an anti-CEA binding domain.

32. The complex of claim 1 or 2, wherein said second ligand binding domain is a chemokine domain.

33. The complex of claim 1 or 2, wherein said second ligand binding domain is an interleukin domain.

34. The complex of claim 1 or 2, wherein said second ligand binding domain is an IL-2 domain, an IL-4 domain, an IL-7 domain, an IL-9 domain, an IL-15 domain, an IL-21 domain or a thymic stromal lymphopoietin (TSLP) domain.

35. The complex of claim 1 or 2, wherein said second ligand binding domain enhancer is a chemokine domain enhancer.

36. The complex of claim 1 or 2, wherein said second ligand binding domain enhancer is an interleukin domain enhancer.

37. The complex of claim 1 or 2, wherein said second ligand binding domain enhancer comprises a sushi domain.

38. The complex of claim 1 or 2, wherein said second ligand binding domain enhancer is an IL-2 domain enhancer, an IL-4 domain enhancer, an IL-7 domain enhancer, an IL-9 domain enhancer, an IL-15 domain enhancer, an IL-21 domain enhancer or a thymic stromal lymphopoietin (TSLP) domain enhancer.

39. The complex of claim 1 or 2, wherein said first chemical linker is a peptidyl linker.

40. The complex of claim 1 or 2, wherein said second chemical linker is a peptidyl linker.

41. The complex of claim 1 or 2, wherein said first chemical linker and said second chemical linker are independently a covalent linker or a non-covalent linker.

42. The complex of claim 1 or 2, wherein said first chemical linker and said second chemical linker are independently a cleavable peptide linker.

43. The complex of claim 1 or 2, wherein said first chemical linker and said second chemical linker are independently an enzymatically cleavable linker.

44. The complex of claim 1 or 2, wherein said first chemical linker and said second chemical linker are independently a protease cleavable linker.

45. The complex of claim 1 or 2, wherein said first chemical linker and said second chemical linker are independently a tumor-associated protease cleavable linker.

46. The complex of claim 1 or 2, wherein said first chemical linker and said second chemical linker independently have a length of about 0 to about 15 amino acid residues.

47. The complex of claim 1 or 2, wherein said first chemical linker and said second chemical linker independently comprise a BSA binding moiety.

48. A pharmaceutical composition comprising a complex of claim 1 or 2 and a pharmaceutically acceptable excipient.

49. A nucleic acid composition comprising a sequence encoding a complex of claim 1 or 2.

50. A cell bound to a complex of claim 1 or 2.

51. The cell of claim 50, wherein said cell is a cancer cell.

52. The cell of claim 50, wherein said cell is an immune cell.

53. A method of treating cancer, said method comprising to a subject in need thereof a therapeutically effective amount of a complex of claim 1 or 2.

54. A covalent complex comprising a ligand binding domain covalently bound to a ligand binding domain enhancer through one or more disulfide linkages.

55. The covalent complex of claim 54, wherein said ligand binding domain is an interleukin domain.

56. The covalent complex of claim 54, wherein said ligand binding domain is an IL-2 domain, an IL-4 domain, an IL-7 domain, an IL-9 domain, an IL-15 domain, an IL-21 domain or a thymic stromal lymphopoietin (TSLP) domain.

57. The covalent complex of claim 56, wherein said IL-15 domain comprises the sequence of SEQ ID NO:42.

58. The covalent complex of claim 56, wherein said IL-2 domain comprises the sequence of SEQ ID NO:40.

59. The covalent complex of claim 54, wherein said ligand binding domain enhancer is a chemokine domain enhancer.

60. The covalent complex of claim 54, wherein said ligand binding domain enhancer is an interleukin domain.

61. The covalent complex of claim 54, wherein said ligand binding domain enhancer is a sushi domain.

62. The covalent complex of claim 54, wherein said ligand binding domain enhancer is an IL-2 domain enhancer, an IL-4 domain enhancer, an IL-7 domain enhancer, an IL-9 domain enhancer, an IL-15 domain enhancer, an IL-21 domain enhancer or a thymic stromal lymphopoietin (TSLP) domain enhancer.

63. The covalent complex of claim 62, wherein said IL-15 domain enhancer comprises the sequence of SEQ ID NO:43.

64. The covalent complex of claim 62, wherein said IL-15 domain enhancer comprises the sequence of SEQ ID NO:44.

65. The covalent complex of claim 62, wherein said IL-2 domain enhancer comprises the sequence of SEQ ID NO:41.

66. The covalent complex of claim 54, wherein said ligand binding domain comprises a cysteine at a position corresponding to position 90 of the sequence of SEQ ID NO:42.

67. The covalent complex of claim 54, wherein said ligand binding domain enhancer comprises a cysteine at a position corresponding to position 67 of the sequence of SEQ ID NO:43.

68. A pharmaceutical composition comprising a complex of claim 54 and a pharmaceutically acceptable excipient.

69. A nucleic acid composition comprising a sequence encoding a complex of claim 54.

70. A cell bound to a covalent complex of claim 54.

71. The cell of claim 70, wherein said cell is a cancer cell.

72. The cell of claim 70, wherein said cell is an immune cell.

73. A method of treating cancer, said method comprising to a subject in need thereof a therapeutically effective amount of a complex of claim 54.


SEQUENCE LISTING (45 sequences)

SEQ ID NO: 1 - protein, 11 aa, Artificial Sequence
  Synthetic Construct
GGVPLSLYSGG

SEQ ID NO: 2 - protein, 11 aa, Artificial Sequence
  Synthetic construct
GGASGSGAGAG

SEQ ID NO: 3 - protein, 11 aa, Artificial Sequence
  Synthetic Construct
GGVPLSLYSGG

SEQ ID NO: 4 - protein, 9 aa, Artificial Sequence
  Synthetic Construct
GGGSGAGAG

SEQ ID NO: 5 - protein, 6 aa, Artificial Sequence
  Synthetic Construct
GGGSGG

SEQ ID NO: 6 - protein, 13 aa, Artificial Sequence
  Synthetic Construct
GGGGVPLSLYSGG

SEQ ID NO: 7 - protein, 6 aa, Artificial Sequence
  Synthetic Construct
GGGSGG

SEQ ID NO: 8 - protein, 13 aa, Artificial Sequence
  Synthetic Construct
GGGGVPLSLYSGG

SEQ ID NO: 9 - protein, 560 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGVPLSLYSGGEVQLVESGGGLVQPGGSLRLSCAASGFNIKDTYIHWVRQSPGKGLEWVARIYPTNGYTRYADSVKGRFTISADTSKNTAYLQMNSLRAEDTAIYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGSGAGAGGMLSLELCDDDPPEIPHATFKAMAYKEGTMLNCECKRGFRRIKSGSLYMLCTGSSSHSSWDNQCQCTSSATRSTTKQVTPQPEEQKERKTTEMCISPMQPVDQASLPGHCREPPPWENEATERIYHFVVGQMVYYQCVQGYRALHRGPAESVCKMTHGKTRWTQPQLICTGEASGGGGHHHHHH

SEQ ID NO: 10 - protein, 214 aa, Artificial Sequence
  Synthetic Construct
DIQMTQSPILLSASVGDRVTITCRASQDVNTAVAWYQQRTNGSPRLLIYSASFLYSGVPSRFSGSRSGTDFTLTISSLQPEDIADYYCQQHYTTPPTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 8 aa, Artificial Sequence
  Synthetic Construct
EVQLVESG

SEQ ID NO: 12 - protein, 31 aa, Artificial Sequence
  Synthetic Construct
GGVPLSLYSGGEVQLVESGGGLVQPGGSLRL

SEQ ID NO: 13 - protein, 31 aa, Artificial Sequence
  Synthetic Construct
GGVPLSLYSGGEVQLVESGGGLVQPGDSLRL

SEQ ID NO: 14 - protein, 31 aa, Artificial Sequence
  Synthetic Construct
GGVPLSLYSGGEVQLVESGGGLVQPGESLRL

SEQ ID NO: 15 - protein, 31 aa, Artificial Sequence
  Synthetic Construct
GGVPLSLYSGGAVQLVESGGGLVQPGGSLRL

SEQ ID NO: 16 - protein, 31 aa, Artificial Sequence
  Synthetic Construct
GGVPLSLYSGGSVQLVESGGGLVQPGGSLRL

SEQ ID NO: 17 - protein, 11 aa, Artificial Sequence
  Synthetic Construct
GGVPLSLYSGG

SEQ ID NO: 18 - protein, 9 aa, Artificial Sequence
  Synthetic Construct
GGGSGAGAG

SEQ ID NO: 19 - protein, 10 aa, Artificial Sequence
  Synthetic Construct
GGSPSGASGS

SEQ ID NO: 20 - protein, 9 aa, Artificial Sequence
  Synthetic Construct
GGGSGAGAG

SEQ ID NO: 21 - protein, 11 aa, Artificial Sequence
  Synthetic Construct
GGVPLSLYSGG

SEQ ID NO: 22 - protein, 9 aa, Artificial Sequence
  Synthetic Construct
GGGSGAGAG

SEQ ID NO: 23 - protein, 10 aa, Artificial Sequence
  Synthetic Construct
GGSPSGASGS

SEQ ID NO: 24 - protein, 9 aa, Artificial Sequence
  Synthetic Construct
GGGSGAGAG

SEQ ID NO: 25 - protein, 10 aa, Artificial Sequence
  Synthetic Construct
TIVPLSLYWN

SEQ ID NO: 26 - protein, 6 aa, Artificial Sequence
  Synthetic Construct
TFPVLV

SEQ ID NO: 27 - protein, 9 aa, Artificial Sequence
  Synthetic Construct
VVPLSLYWN

SEQ ID NO: 28 - protein, 6 aa, Artificial Sequence
  Synthetic Construct
TFPVLV

SEQ ID NO: 29 - protein, 8 aa, Artificial Sequence
  Synthetic Construct
ARLAELNA

SEQ ID NO: 30 - protein, 6 aa, Artificial Sequence
  Synthetic Construct
VPLSLY

SEQ ID NO: 31 - protein, 7 aa, Artificial Sequence
  Synthetic Construct
IVPLSLY

SEQ ID NO: 32 - protein, 6 aa, Artificial Sequence
  Synthetic Construct
TFPVLV

SEQ ID NO: 33 - protein, 9 aa, Artificial Sequence
  Synthetic Construct
VVPLSLYWN

SEQ ID NO: 34 - protein, 9 aa, Artificial Sequence
  Synthetic Construct
VVPLSLYWN

SEQ ID NO: 35 - protein, 8 aa, Artificial Sequence
  Synthetic Construct
VPLSLYFT

SEQ ID NO: 36 - protein, 10 aa, Artificial Sequence
  Synthetic Construct
IQEARERWNF

SEQ ID NO: 37 - protein, 8 aa, Artificial Sequence
  Synthetic Construct
TFPVLVRT

SEQ ID NO: 38 - protein, 10 aa, Artificial Sequence
  Synthetic Construct
TLVPLSLYWK

SEQ ID NO: 39 - protein, 223 aa, Artificial Sequence
  Synthetic Construct
EVQLVESGGGLVQPGGSLRLSCAASGFNIKDTYIHWVRQSPGKGLEWVARIYPTNGYTRYADSVKGRFTISADTSKNTAYLQMNSLRAEDTAIYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 40 - protein, 133 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 41 - protein, 168 aa, Artificial Sequence
  Synthetic Construct
SLELCDDDPPEIPHATFKAMAYKEGTMLNCECKRGFRRIKSGSLYMLCTGSSSHSSWDNQCQCTSSATRSTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 42 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 43 - protein, 78 aa, Homo sapiens
ITCPPPMSVEHADIWVKSYSLYSRERYICNSGFKRKAGTSSLTECVLNKATNVAHWTTPSLKCIRDPALVHQRPAPPS

SEQ ID NO: 44 - protein, 78 aa, Artificial Sequence
  Synthetic Construct
ITCPPPMSVEHADIWVKSYSLYSRERYICNSGFKRKAGTSSLTECVLNKATNVAHWTTPSLKCIRDCALVHQRPAPPS

SEQ ID NO: 45 - protein, 114 aa, Artificial Sequence
  Synthetic Construct
NWVNVISDLKKIEDLIQSMHIDATLYTESDVHPSCKVTAMKCFLLELQVISLESGDASIHDTVENLIILANNSLSSNGNVTESGCKECCELEEKNIKEFLQSFVHIVQMFINTS